(19) United States
(12) Patent Application Publication
(10) Pub. No.: US 2021/0163498 A1
(43) Pub. Date: Jun. 3, 2021

(54) SOLID STATE FORMS OF LORLATINIB AND THEIR PREPARATION

(71) Applicant: PLIVA HRVATSKA D.O.O., Zagreb (HR)

(72) Inventors: Maja Matanovic Skugor, Zagreb (HR); Ivica Grebenar, Zagreb (HR); Nea Baus Topic, Rijeka (HR); Dijana Skalec Samec, Jastrebarsko (HR); Edi Topic, Malinska (HR)

(21) Appl. No.: 17/047,417

(22) PCT Filed: Apr. 19, 2019
(86) PCT No.: PCT/US19/28221

§ 371 Date: Oct. 14, 2020

Related U.S. Application Data
(60) Provisional application No. 62/703,729, filed on Jul. 26, 2018.
(60) Provisional application No. 62/696,437, filed on Jul. 11, 2018.
(60) Provisional application No. 62/677,269, filed on May 29, 2018.
(60) Provisional application No. 62/668,894, filed on May 9, 2018.
(60) Provisional application No. 62/661,175, filed on Apr. 23, 2018.

(51) Int. Cl. C07D 498/18

(52) U.S. Cl. CPC C07D 498/18; C07B 2200/13

(57) ABSTRACT

The present disclosure relates to Lorlatinib solid state forms, Lorlatinib salts and solid states thereof, processes for preparation thereof, pharmaceutical compositions and methods of use thereof.

### FIELD OF THE INVENTION

The present disclosure relates to Lorlatinib solid state forms, Lorlatinib salts and solid states thereof, processes for preparation thereof, pharmaceutical compositions, and methods of use thereof.

### BACKGROUND

Lorlatinib, described also as PF-6463922, has the chemical name (10R)-7-amino-12-fluoro-2,10,16-trimethyl-15-oxo-10,15,16,17-tetrahydro-2H-8,4-(metheno)pyrazolo[4,3-h][2,5,11]benzoxadiazacyclotetradecine-3-carbonitrile. Lorlatinib is developed for the treatment of cancer, mainly treating non-small cell lung cancer. Lorlatinib has the following chemical structure:

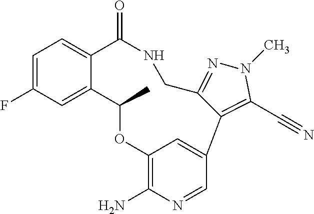 [SMILES: C[C@H]1Oc2cc(cnc2N)-c2c(nn(C)c2C#N)CNC(=O)c2ccc(F)cc21]

Lorlatinib is disclosed in U.S. Pat. Nos. 8,680,111 and 9,133,215. Solid state forms of Lorlatinib are disclosed in U.S. Pat. No. 9,637,500 (referred to therein as forms 1, 2 & 3) and in International Publication No. WO 2017/021823.

Lorlatinib salts are also described in the literature. Lorlatinib hydrochloride salt is described in J. Med. Chem. 2014, 57, 4720-4744 (designated as compound 8K) and Lorlatinib maleate salt, forms I and II, is described in International Publication No. WO 2017/175091.

Polymorphism, the occurrence of different crystal forms, is a property of some molecules and molecular complexes. A single compound, like Lorlatinib, may give rise to a variety of polymorphs having distinct crystal structures and physical properties like melting point, thermal behaviors (e.g. measured by thermogravimetric analysis—“TGA”, or differential scanning calorimetry—“DSC”), X-ray powder diffraction (XRPD) pattern, infrared absorption fingerprint, Raman absorption fingerprint, and solid state (¹³C-) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms may provide a basis for improving formulation, for example, by facilitating better processing or handling characteristics, improving the dissolution profile, or improving stability (polymorph as well as chemical stability) and shelf-life. These variations in the properties of different salts and solid state forms may also provide improvements to the final dosage form, for instance, if they serve to improve bioavailability. Different salts and solid state forms of an active pharmaceutical ingredient may also give rise to a variety of polymorphs or crystalline forms, which may in turn provide additional opportunities to use variations in the properties and characteristics of a solid active pharmaceutical ingredient for providing an improved product.

Discovering new salts and solid state forms of a pharmaceutical product can provide materials having desirable processing properties, such as ease of handling, ease of processing, storage stability, and ease of purification, or as desirable intermediate crystal forms that facilitate conversion to other salts or polymorphic forms. New polymorphic forms and new salts of a pharmaceutically useful compound can also provide an opportunity to improve the performance characteristics of a pharmaceutical product (dissolution profile, bioavailability, etc.). It enlarges the repertoire of materials that a formulation scientist has available for formulation optimization, for example by providing a product with different properties, e.g., a different crystal habit, higher crystallinity or polymorphic stability, which may offer better processing or handling characteristics, improved dissolution profile, or improved shelf-life. For at least these reasons, additional salts and solid state forms of Lorlatinib are desirable.

### SUMMARY OF THE INVENTION

The present disclosure relates to Lorlatinib solid state forms, Lorlatinib salts and their solid state forms thereof, to processes for preparation thereof, and to pharmaceutical compositions including these salts and solid state forms.

In embodiments, the present disclosure provides crystalline forms of Lorlatinib designated as Forms Z, U1, U2, Gamma, Epsilon, X, E1 and E2 (defined herein). In some embodiments, Form X and Form Epsilon (defined herein) are provided.

In embodiments, the present disclosure provides Lorlatinib salts and crystalline forms thereof. In embodiments, the present disclosure provides Lorlatinib Fumarate (Form F1), Lorlatinib Benzoate (Form B1), Lorlatinib Nicotinate (Form N1), Lorlatinib Mesylate (Form S1), Lorlatinib Tosylate (Form T1), Lorlatinib Hydrobromide (Form H1), Lorlatinib L-Malate (Form L1), Lorlatinib Citrate (Form C1), Lorlatinib L-Tartarate (Form R1) and Lorlatinib Maleate (Forms M1, M2, M4 and M5).

The present disclosure further provides processes for preparing Lorlatinib and Lorlatinib salts and solid state forms thereof.

The present disclosure also relates to the uses of any one or combinations of the solid state forms of Lorlatinib and salts thereof of the present disclosure, for preparing other solid state forms of Lorlatinib and Lorlatinib salts and their solid state forms thereof.

The present disclosure also relates to any one or a combination of the solid state forms of Lorlatinib and Lorlatinib salts, including Lorlatinib hydrochloride of the present disclosure, for use in preparing other solid state forms of Lorlatinib and Lorlatinib salts and their solid state forms thereof.

In another aspect, the present disclosure encompasses the above described solid state forms of Lorlatinib and Lorlatinib salts and solid state forms thereof for use in the preparation of pharmaceutical compositions and/or formulations, in embodiments for use in medicine, including for treatment of non-small cell lung cancer.

In another aspect, the present disclosure encompasses the use of the above described solid state forms of Lorlatinib and Lorlatinib salts and solid state forms thereof for the preparation of pharmaceutical compositions and/or formulations, in embodiments for use in medicine, including for treatment of non-small cell lung cancer.

In yet another embodiment, the present disclosure encompasses pharmaceutical compositions including any one or a mixture of the solid state forms of Lorlatinib and Lorlatinib salts and solid state forms thereof.

In an embodiment, the present disclosure encompasses pharmaceutical formulations including any one or a mixture of the solid state forms of Lorlatinib and Lorlatinib salts and solid state forms thereof, and at least one pharmaceutically acceptable excipient.

The present disclosure further encompasses processes to prepare said pharmaceutical formulations of Lorlatinib and/or salts of Lorlatinib, by combining any one or a mixture of the above described salts and solid state forms of Lorlatinib, or pharmaceutical compositions including them, and at least one pharmaceutically acceptable excipient.

The solid state forms of Lorlatinib and Lorlatinib salts and their solid state forms thereof as defined herein, as well as the pharmaceutical compositions or formulations including them, can be used as medicaments, in embodiments for treating non-small cell lung cancer, by administering a therapeutically effective amount of any of the solid state forms and/or salts of the present disclosure, or at least one of the above pharmaceutical compositions or formulations, to a subject suffering from cancer, or otherwise in need of the treatment.

The present disclosure also provides the uses of the solid state forms of Lorlatinib and Lorlatinib salts and their solid state forms thereof of the present disclosure, or at least one of the above pharmaceutical compositions or formulations, for the manufacture of medicaments for treating cancer, in embodiments for treating non-small cell lung cancer.

### BRIEF DESCRIPTION OF THE DRAWINGS

FIG. 1 shows an X-ray powder diffraction (XRPD) pattern of Lorlatinib Form Z.

FIG. 2 shows an XRPD pattern of Lorlatinib Form Gamma.

FIG. 3 shows an XRPD pattern of Lorlatinib Form U1.

FIG. 4 shows an XRPD pattern of Lorlatinib Form U2.

FIG. 5 shows an XRPD pattern of Amorphous Lorlatinib.

FIG. 6 shows an XRPD pattern of Lorlatinib Form 2, as described in U.S. Pat. No. 9,637,500 (FIG. 2).

FIG. 7 shows an XRPD pattern of Lorlatinib Form 1, as described in U.S. Pat. No. 9,637,500 (FIG. 1).

FIG. 8 shows the XRPD pattern of (10R)-(7-di-tert-butyloxycarbonylamino)-12-fluoro-2,10,16-trimethyl-15-oxo-10,15,16,17-tetrahydro-2H-8,4-(metheno)pyrazolo[4,3-h]-[2,5,11]benzoxadiazacyclo-tetradecine-3-carbonitrile.

FIG. 9 shows the XRPD pattern of Lorlatinib Form Epsilon.

FIG. 10 shows the XRPD pattern of Lorlatinib Fumarate Form F1.

FIG. 11 shows the XRPD pattern of Lorlatinib Benzoate Form B1.

FIG. 12 shows the XRPD pattern of Lorlatinib Nicotinate Form N1.

FIG. 13 shows the XRPD pattern of Lorlatinib Mesylate Form S1.

FIG. 14 shows the XRPD pattern of Lorlatinib Tosylate Form T1.

FIG. 15 shows the XRPD pattern of Lorlatinib Hydrobromide form H1.

FIG. 16 shows the XRPD pattern of Lorlatinib L-Malate form L1.

FIG. 17 shows the XRPD pattern of Lorlatinib Citrate Form C1.

FIG. 18 shows the XRPD pattern of Lorlatinib L-Tartarate form R1.

FIG. 19 shows the XRPD pattern of Lorlatinib Form X.

FIG. 20 shows the XRPD pattern of Lorlatinib Form E1.

FIG. 21 shows the XRPD pattern of Lorlatinib Form E2.

FIG. 22 shows the XRPD pattern of Lorlatinib Maleate Form M1.

FIG. 23 shows the XRPD pattern of Lorlatinib Maleate Form M2.

FIG. 24 shows the XRPD pattern of Lorlatinib Maleate Form M4.

FIG. 25 shows the XRPD pattern of Lorlatinib Maleate Form M5.

FIG. 26 shows the XRPD pattern of Lorlatinib Maleate Form 1 (described in International Publication No. WO 2017/175091).

FIG. 27 shows solid state ¹³C-SS NMR spectrum of Lorlatnib Form X at the range of 200-0 ppm.

FIG. 28 shows solid state ¹³C-SS NMR spectrum of Lorlatinib Form X at the range of 200-100 ppm.

FIG. 29 shows solid state ¹³C-SS NMR spectrum of Lorlatinib Form X at the range of 100-0 ppm.

FIG. 30 shows Raman spectrum of Lorlatinib Form X at the range of 500-4000 cm-1.

FIG. 31 shows FTIR spectrum of Lorlatinib Form X at the range of 500-4000 cm-1.

FIG. 32 shows DSC thermogram of Lorlatinib Form X measured at the temperature range of 25-300° C.

FIG. 33 shows solid state ¹³C-SS NMR spectrum of Lorlatinib Form Epsilon at the range of 200-0 ppm.

FIG. 34 shows solid state ¹³C-SS NMR spectrum of Lorlatinib Form Epsilon at the range of 200-100 ppm.

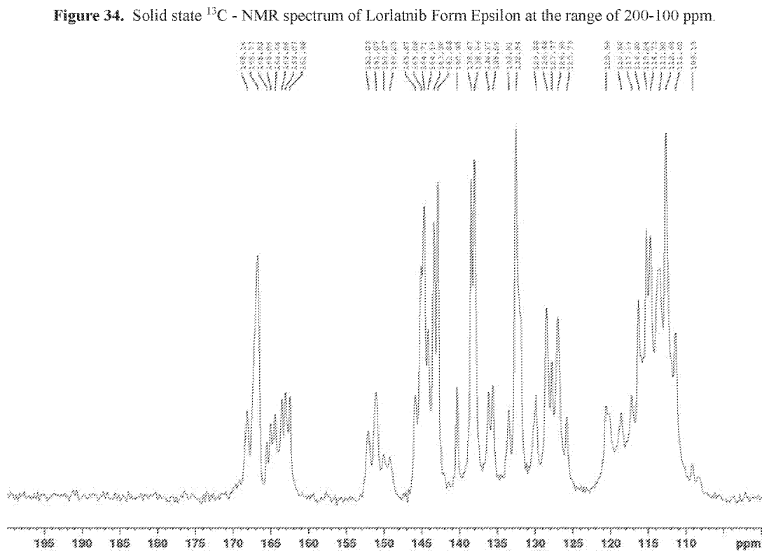

FIG. 35 shows solid state ¹³C-SS NMR spectrum of Lorlatinib Form Epsilon at the range of 100-0 ppm.

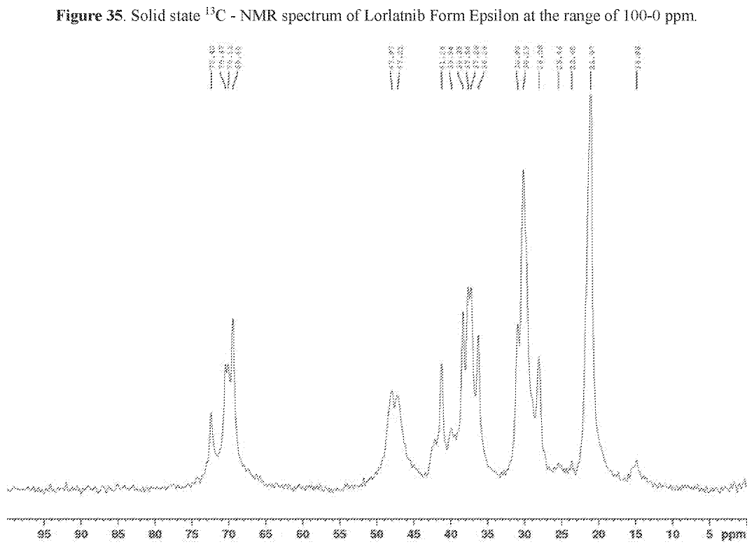

### DETAILED DESCRIPTION OF ILLUSTRATIVE EMBODIMENTS

The present disclosure relates to solid state forms of Lorlatinib, Lorlatinib salts and their solid states thereof, to processes for preparation thereof and to pharmaceutical compositions including these salts and solid state forms and/or combinations thereof. The disclosure also relates to the conversion of the solid state forms of Lorlatinib to other solid state forms of Lorlatinib and/or to Lorlatinib salts, and to the conversion of Lorlatinib salt, such as hydrochloride, to other solid state forms of Lorlatinib and/or to Lorlatinib salts.

The Lorlatinib solid state forms and salts thereof according to the present disclosure may have advantageous properties selected from at least one of: chemical or polymorphic purity, flowability, solubility, dissolution rate, bioavailability, morphology or crystal habit, stability, such as chemical stability as well as thermal and mechanical stability with respect to polymorphic conversion, stability towards dehydration and/or storage stability, a lower degree of hygroscopicity, low content of residual solvents, adhesive tendencies and advantageous processing and handling characteristics such as compressibility, and bulk density.

A crystal form may be referred to herein as being characterized by graphical data “as depicted in” a Figure. Such data include, for example, powder X-ray diffractogram and solid state NMR spectra. As is well-known in the art, the graphical data potentially provides additional technical information to further define the respective solid state form (a so-called “fingerprint”) which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms.

A crystal form of Lorlatinib referred to herein as being characterized by graphical data “as depicted in” a Figure will thus be understood to include any crystal form of Lorlatinib, characterized with the graphical data having such small variations, as are well known to the skilled person, in comparison with the Figure.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% (w/w) of any other forms of the subject compound as measured, for example, by XRPD. Thus, solid states of Lorlatinib or a salt thereof described herein as substantially free of any other solid state forms including other solid state forms of salts would be understood to contain greater than about 80% (w/w), greater than about 90% (w/w), greater than about 95% (w/w), greater than about 98% (w/w), greater than about 99% (w/w), or about 100% of the subject salts or solid state form of Lorlatinib. Accordingly, in some embodiments of the disclosure, the described salts and solid state forms of Lorlatinib may contain from about 1% to about 20% (w/w), from about 5% to about 20% (w/w), or from about 5% to about 10% (w/w) of one or more solid state forms of Lorlatinib or salts thereof.

As used herein, unless stated otherwise, XRPD peaks reported herein are measured using CuKα radiation, λ=1.54184 Å, in embodiments, XRPD peaks reported herein are measured using CuK α radiation, λ=1.54184 Å, at a temperature of 25±3° C.

As used herein, unless stated otherwise, ¹³C-SS solid state NMR was measured at 125.77 MHz and 303 K at a spin rate of 18 kHz.

As used herein, unless stated otherwise, Raman spectrum was recorded on an FT-IR spectrometer with FT-Raman module equipped with a 1064 nm excitation laser, CaF2 beam splitter and a Ge detector at spectral resolution of 4.0 cm⁻¹.

As used herein, unless stated otherwise, IR spectrum was recorded on an FT-IR spectrometer equipped with KBr beamsplitter at a resolution of 4.0 cm⁻¹.

As used herein, unless otherwise, DSC measurement was done at a heating rate of 10° C./min.

As used herein, the term “isolated” in reference to solid state forms and salts of Lorlatinib of the present disclosure corresponds to salts and solid state forms of Lorlatinib that are physically separated from the reaction mixture in which they are is formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

A process or step may be referred to herein as being carried out “overnight.” This refers to a time interval, e.g., for the process or step, that spans the time during the night, when that process or step may not be actively observed. This time interval is from about 8 to about 20 hours, or about 10 to about 18 hours, typically about 16 hours.

As used herein, and unless stated otherwise, the term “anhydrous” is in relation to crystalline Lorlatinib or a salt thereof which does not include any crystalline water (or other solvents) in a defined, stoichiometric amount within the crystal. Moreover, an “anhydrous” form does not contain more than about 1% (w/w) of either water or organic solvents as measured, for example, by TGA, Karl Fischer, or by other suitable technique.

The term “solvate”, as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding (methyl tert-butyl ether) MTBE (1.5 v/v) to a 100 ml reaction mixture would indicate that 150 mL of MTBE was added.

Preferably, unless otherwise indicated, percentages relate to weight percent (wt %).

As used herein the term non-hygroscopic in relation to crystalline Lorlatinib or a salt thereof, refers to less than about 1.0% (w/w) absorption of water at about 25° C. and about 80% relative humidity (RH), as determined for example by TGA or other suitable technique.

As used herein, and unless indicated otherwise, the term “thermo-dynamical stability” in relation to solid state forms of Lorlatinib and salts thereof refers to resistance of the solid state form to polymorphic conversion under certain conditions, for example, heating, melting or dissolving. In some embodiments, the term refers to less than about 20% (w/w), about 10% (w/w), about 5% (w/w), about 1% (w/w), about 0.5% (w/w), or about 0% (w/w) conversion of crystalline Lorlatinib or a salt thereof to any other solid state form of Lorlatinib or a salt thereof as measured by XRPD. In some embodiments, the conversion is about 1% (w/w) to about 20% (w/w), about 1% (w/w) to about 10% (w/w) or about 1% (w/w) to about 5% (w/w).

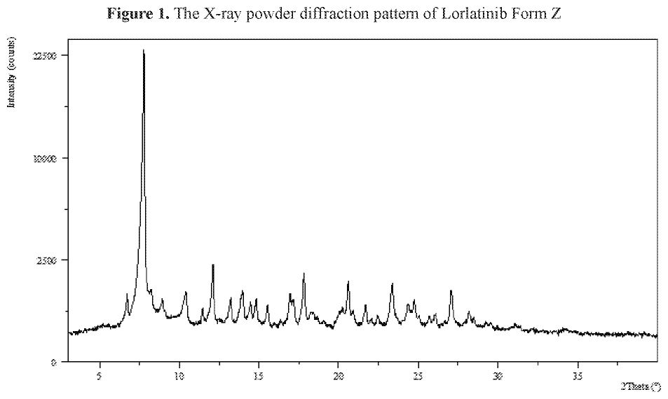

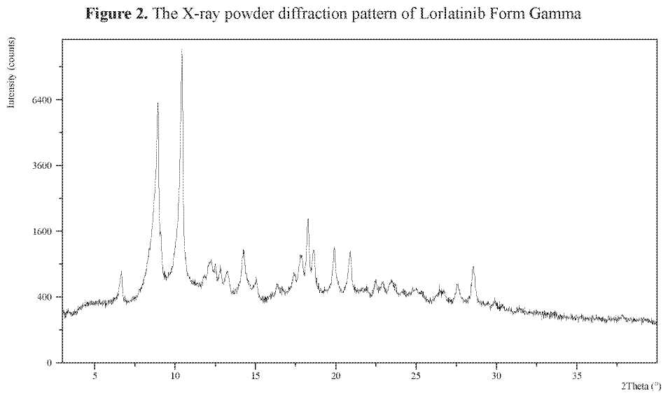

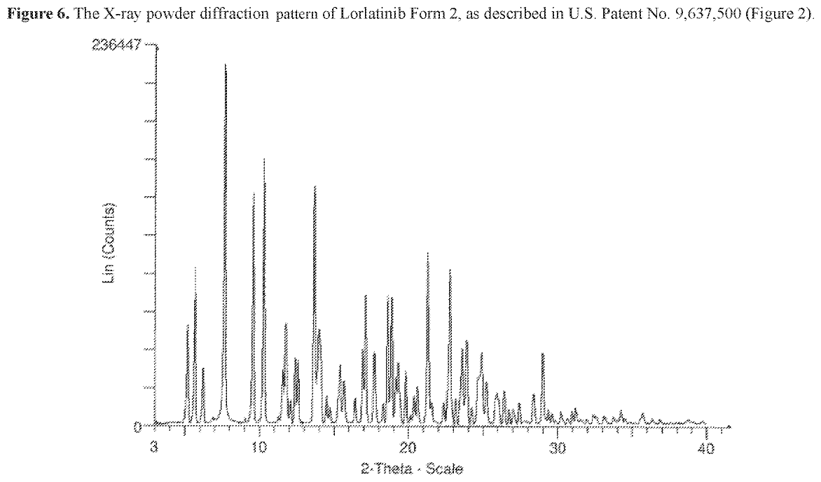

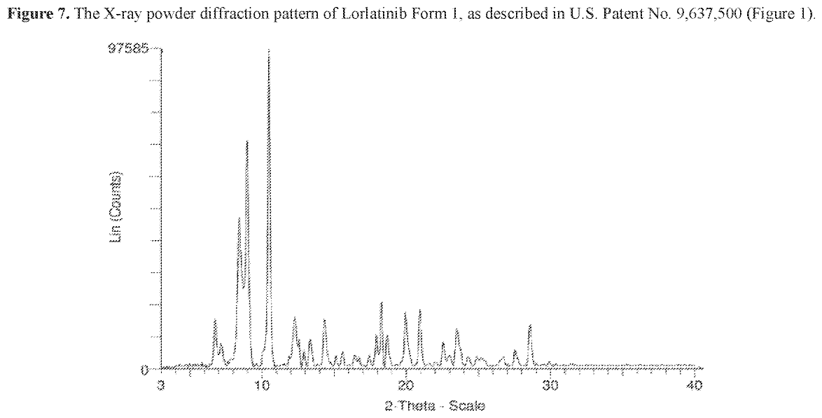

As used therein the terms “Lorlatinib Form 1” and “Lorlatinib Form 2” relate to crystalline form having an XRPD pattern shown herein in FIGS. 7 and 6 respectfully, as described in U.S. Pat. No. 9,637,500 (FIGS. 1 and 2 therein).

As used herein the term “Lorlatinib Hydrochloride” relates to Lorlatinib hydrochloride salt and its preparation as described in the J. Med. Chem. 2014, 57, 4720-4744.

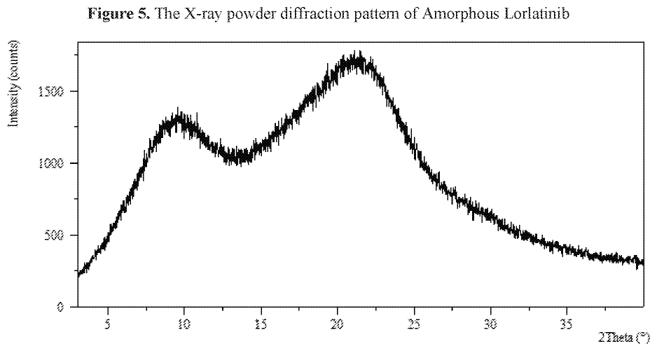

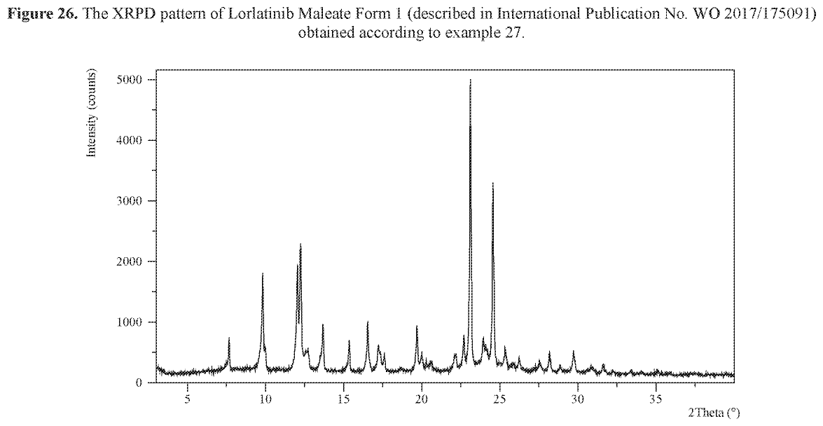

As used herein the term “Lorlatinib Maleate form 1” relates to crystalline form having an XRPD pattern shown herein in FIG. 26, as described in International Publication No. WO 2017/175091 (FIG. 5 therein).

The present disclosure includes a crystalline form of Lorlatinib designated as Form Z. The crystalline Form Z of Lorlatinib can be characterized by data selected from one or more of the following: an XRPD pattern having peaks at 13.2, 15.6, 23.4, 24.7 and 27.0 degrees 2-theta±0.2 degrees 2-theta; an XRPD pattern substantially as depicted in FIG. 1; or combinations of these data.

Lorlatinib Form Z may be characterized as hydrate form. In embodiments, it may be characterized as dihydrate. In certain embodiments, Form Z may contain from about 6% to about 11% of water by weight, in embodiments from about 7% to about 10% by weight, in other embodiments from about 7% to about 9%, as determined for example by Karl Fischer or by other suitable techniques (the theoretical water content for dihydrate is about 8.3%).

The present disclosure includes a crystalline form of Lorlatinib designated as Form Gamma. The crystalline Form Gamma of Lorlatinib can be characterized by data selected from one or more of the following: an XRPD pattern having peaks at 6.7, 8.9, 10.4, 12.3 and 14.3 degrees two theta±0.2 degrees two-theta and absence of peaks at 7.1, 8.4 and 15.6 degrees two-theta±0.2 degrees 2-theta; an XRPD pattern substantially as depicted in FIG. 2; or combinations of these data.

Crystalline Lorlatinib Form Gamma may be characterized as hydrate. In embodiments, it may be characterized as hemihydrate. Lorlatinib Form Gamma may contain about 1-3% of water, as determined for example by Karl Fischer or by other suitable techniques (the theoretical water content for hemihydrate is 2%).

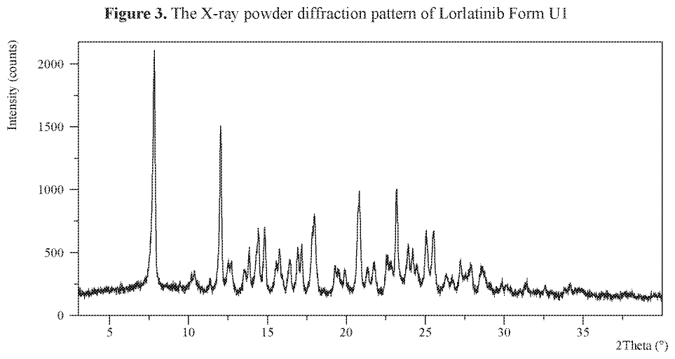

The present disclosure includes a crystalline form of Lorlatinib designated as Form U1. The crystalline Form U1 of Lorlatinib can be characterized by data selected from one or more of the following: an XRPD pattern having peaks at 12.0, 12.7, 13.5, 13.8 and 14.5 degrees 2-theta±0.2 degrees 2-theta; an XRPD pattern substantially as depicted in FIG. 3; or combinations of these data. Form U1 may be characterized as hydrate.

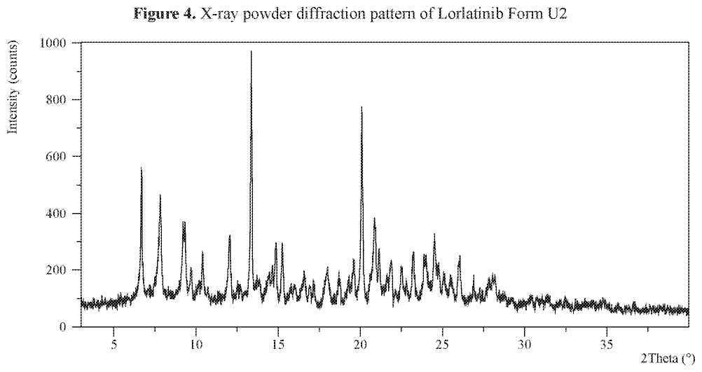

The present disclosure includes a crystalline form of Lorlatinib designated as Form U2. The crystalline Form U2 of Lorlatinib can be characterized by data selected from one or more of the following: an XRPD pattern having peaks at 6.7, 13.4, 18.7, 20.1 and 26.0 degrees 2-theta±0.2 degrees 2-theta; an XRPD pattern substantially as depicted in FIG. 4; or combinations of these data. Form U2 may be characterized as hydrate.

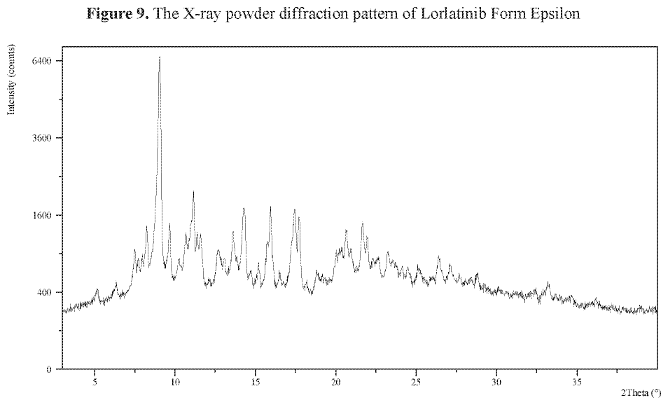

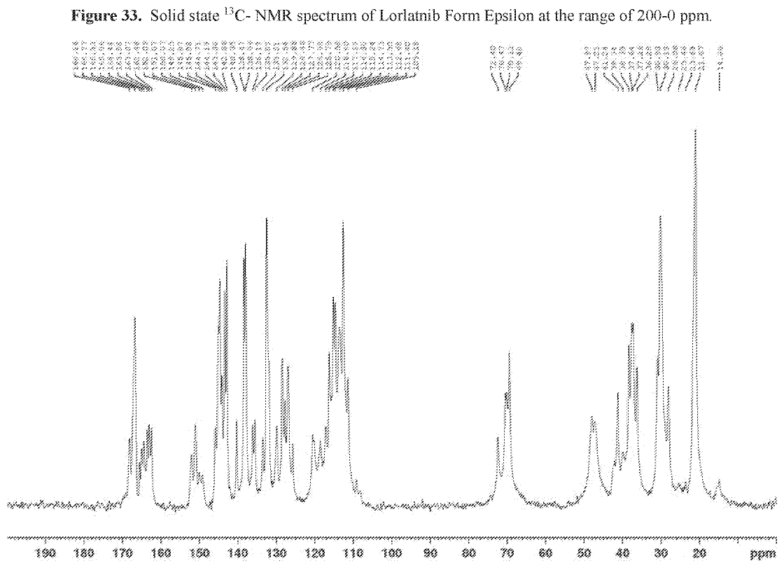

The present disclosure includes a crystalline form of Lorlatinib designated as Form Epsilon. The crystalline Form Epsilon of Lorlatinib can be characterized by data selected from or more of the following: an XRPD pattern having peaks at 6.4, 10.7, 9.0, 13.1 and 13.6 degrees two theta±0.2 degrees two-theta; an XRPD pattern substantially as depicted in FIG. 9; a solid-state ¹³C-SS NMR spectrum with signals at 21.1, 38.3, 41.2, 115.2 and 132.5 ppm±0.2 ppm; a solid state ¹³C-SS NMR spectrum having the following chemical shift absolute differences from a peak at 21.1 ppm±2 ppm of 0, 17.2, 20.1, 94.1 and 111.4 ppm±0.1 ppm; a solid-state ¹³C-SS NMR spectrum as depicted in FIG. 33, 34 or 35; or combinations of these data.

Crystalline Lorlatinib Form Epsilon may be further characterized by data selected from one or more of the following: an XRPD pattern having peaks at 6.4, 10.7, 9.0, 13.1 and 13.6 degrees two theta±0.2 degrees two-theta; and also having one, two, three, four or five additional peaks selected from 7.7, 8.2, 20.7, 21.7 and 23.7 degrees two theta±0.2 degrees two theta; or combinations of these data.

Alternatively, crystalline Form Epsilon of Lorlatinib can be characterized by data selected from or more of the following: an XRPD pattern having peaks at 6.4, 10.7, 9.0, 13.1 and 13.6 degrees two theta±0.2 degrees two-theta and also having one, two, three, four or five additional peaks selected from 7.7, 8.2, 20.7, 21.7 and 23.7 degrees two theta±0.2 degrees two theta and further having an absence of at least one (in embodiments both) of the peaks at 6.7 and 7.2±0.2 degrees two-theta; an XRPD pattern substantially as depicted in FIG. 9; a solid-state ¹³C-SS NMR spectrum with signals at 21.1, 38.3, 41.2, 115.2 and 132.5 ppm±0.2 ppm; a solid state ¹³C-SS NMR spectrum having the following chemical shift absolute differences from a peak at 21.1 ppm±2 ppm of 0, 17.2, 20.1, 94.1 and 111.4 ppm±0.1 ppm; a solid-state ¹³C-SS NMR spectrum as depicted in FIG. 33, 34 or 35; or combinations of these data.

Lorlatinib Form Epsilon as described in any embodiment herein can be characterized as a hydrate form containing about 1-4% by weight of water, in embodiments about 2-3% by weight of water, in some embodiments about 3% by weight of water as measured by any suitable technique, e.g. by Karl Fischer.

Crystalline Lorlatinib Form Epsilon may alternatively be characterized by an XRPD pattern having peaks at 6.4, 7.7, 8.2, 10.7, 9.0, 13.1, 13.6, 20.7, 21.7 and 23.7 degrees two theta±0.2 degrees two theta. Alternatively, crystalline Lorlatinib Form Epsilon may be characterized by an XRPD pattern having peaks at 6.4, 7.7, 8.2, 10.7, 9.0, 13.1, 13.6, 20.7, 21.7 and 23.7 degrees two theta±0.2 degrees two theta and further having an absence of at least one (in embodiments both) of the peaks at 6.7 and 7.2±0.2 degrees two-theta.

Crystalline Lorlatinib Form Epsilon may be characterized by each of the above characteristics alone/or by all possible combinations, e.g. by XRPD pattern having peaks at 6.4, 10.7, 9.0, 13.1 and 13.6 degrees two theta±0.2 degrees two-theta and an XRPD pattern substantially as depicted in FIG. 9, and combinations thereof, or by an XRPD pattern having peaks at 6.4, 7.7, 8.2, 10.7, 9.0, 13.1, 13.6, 20.7, 21.7 and 23.7 degrees two theta±0.2 degrees two theta and further having an absence of at least one (in embodiments both) of the peaks at 6.7 and 7.2±0.2 degrees two-theta and an XRPD pattern substantially as depicted in FIG. 9.

In any aspect or embodiment of the present disclosure, the crystalline Lorlatinib Form Epsilon described herein may be substantially free of any other solid state forms of Lorlatinib. In embodiments, crystalline Lorlatinib Form Epsilon according to any aspect or embodiment of the present disclosure contains about 20% (w/w) or less, about 10% (w/w) or less, about 5% (w/w) or less, about 2% (w/w) or less, about 1% (w/w) or less, or about 0% (w/w) of any other forms of Lorlatinib as measured, for example, by XRPD. Thus, according to any aspect or embodiment of the present disclosure, the crystalline form includes greater than about 80% (w/w), greater than about 90% (w/w), greater than about 95% (w/w), greater than about 98% (w/w), greater than about 99% (w/w), or about 100% of Form Epsilon of Lorlatinib. In embodiments, Lorlatinib Form Epsilon according to any aspect or embodiment of the present disclosure contains no detectable amounts of any other solid state forms of Lorlatinib (for example, as measured by XRPD).

In another embodiment of the present disclosure, crystalline Lorlatinib Form Epsilon is polymorphically pure.

In another embodiment of the present disclosure, crystalline Lorlatinib Form Epsilon is isolated.

As described above, depending on which other solid state it is compared with, Form Epsilon of Lorlatinib according to the present disclosure may have advantageous properties as described above. In embodiments, Lorlatinib Form Epsilon is polymorphically pure containing very low residual solvent, in embodiments: ≤1 wt %, ≤0.5 wt %, ≤0.2 wt %, ≤0.1 wt %, ≤0.05 wt %, ≤0.025 wt %, ≤0.0125 wt %, of residual solvent. Residual solvents in this context refer to organic solvents that are not part of the crystal structure, for example C₁-C₆ alcohols, including, methanol. In embodiments, Lorlatinib Form Epsilon as described in any aspect or embodiment contains: ≤1 wt %, ≤0.5 wt %, ≤0.2 wt %, ≤0.1 wt %, ≤0.05 wt % or ≤0.025 wt, %≤0.0125 wt %, or ≤0.01 wt % of residual solvent. In other embodiments the Lorlatinib Form Epsilon as defined in any of the above embodiments contains: ≤1 wt %, ≤0.5 wt %, ≤0.2 wt %, ≤0.1 wt %, ≤0.05 wt %, ≤0.025 wt, %≤0.0125 wt %, or ≤0.01 wt % of methanol as residual solvent.

The above described Lorlatinib Form Epsilon can be prepared by a process including crystallization of Lorlatinib in methanol. In embodiments wherein the Form Epsilon is a hydrate, the crystallisation process is carried out without excluding water. Thus, without being bound by theory, in the herein described processes for preparing Lorlatinib Form Epsilon in the form of a hydrate, the water for the hydrate may be present in the atmosphere, as trace or residual amounts in the methanol or may be present in the starting material (e.g., a hydrated form of the starting material).

In embodiments, the process includes using any one or a mixture of Lorlatinib solid state forms and/or any one or a mixture of Lorlatinib salts and their solid state forms thereof, such as Lorlatinib hydrochloride.

In embodiments, Lorlatinib salt is first converted to Lorlatinib. In embodiments, Lorlatinib salt is converted in a suitable solvent with a suitable base. In embodiments, any Lorlatinib salt can be used, such as Lorlatinib hydrochloride. In embodiments, a suitable solvent may include a polar aprotic solvent, such as dichloromethane, ethyl acetate, n-butyl acetate, toluene, isobutyl acetate and 2-methyl THF, with dichloromethane used in some embodiments. In embodiments, a suitable base is selected from inorganic and organic base, such as sodium hydroxide, potassium hydroxide, sodium carbonate, sodium bicarbonate, potassium carbonate and potassium bicarbonate, with sodium hydroxide used in some embodiments. In certain embodiments, the Lorlatinib salt is basified with an aqueous inorganic base in the presence of the suitable organic solvent which is not miscible with water.

In embodiments, isolation of Lorlatinib obtained in previous stage may be done, for example, by extraction of the Lorlatinib free base into the organic solvent, followed by solvent evaporation. In some embodiments, the organic solvent is dichloromethane. In other embodiments, the solvent is evaporated until dryness to provide Lorlatinib free base.

Preparation of Lorlatinib Form Epsilon may be carried out by crystallization of Lorlatinib free base in methanol. In some embodiments the crystallization is carried out by dissolution of the Lorlatinib in methanol, and optionally cooling the solution. In embodiments, 1-8 vol of methanol, in some embodiments 2-6 vol, in other embodiments 2-5 vol, and in yet other embodiments about 3 to about 4 vol of methanol may be used to dissolve Lorlatinib.

The prepared solution may be kept at room temperature, in embodiments at about 20° C. to about 25° C., in some embodiments at 25° C., optionally with stirring for about several minutes until precipitation is observed.

Optionally, the slurry is further kept under the above described conditions for about 1-5 hours, in some embodiments for about 1-3 hours, in yet other embodiments for about 2 hours, optionally with stirring.

Optionally, the suspension may be cooled to temperature of about 0 to about 20° C., in embodiments about 0 to about 10° C., in some embodiments about 0 to 5° C. The cooling can be carried out over about 20 to 180 minutes, in embodiments for about 25 to about 120 minutes, in embodiments for about 25 to 60 minutes, in yet other embodiments for about 30 minutes, optionally with stirring.

Optionally the suspension is held at the cooling temperature for an additional 30 to about 180 minutes, in embodiments for about an additional 60 to about 150 minutes, in embodiments for about 120 minutes, optionally with stirring.

Isolation of the said crystalline form may be done for example by filtering the suspension and drying. Drying may be done by nitrogen or air or under vacuum. In embodiments, drying is performed at a temperature of about 40-60° C., in embodiments about 40-50° C., and in other embodiments about 45° C., in some cases under vacuum (in embodiments a reduced pressure of: 1-200 mbar, in other embodiments 1-100 mbar, in some embodiments 1-50 mbar, and in other embodiments 1-20 mbar or about 10 mbar).

The process may further include combining the crystalline Lorlatinib Form Epsilon with at least one pharmaceutically acceptable excipient to provide a pharmaceutical composition or formulation.

The process disclosure also relates to a crystalline form of Lorlatinib Form Epsilon or a pharmaceutical composition or formulation thereof, which is obtainable by any embodiment of the process as described above.

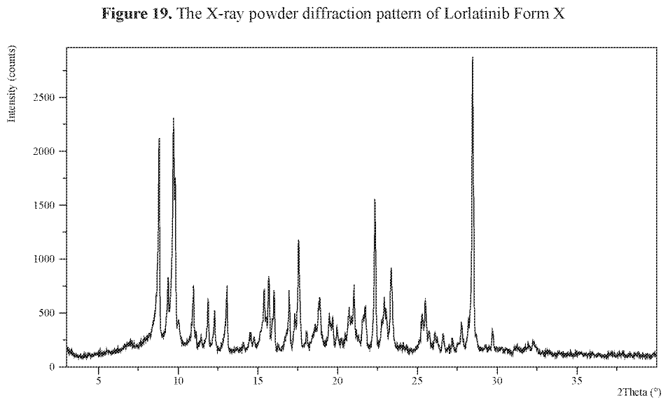

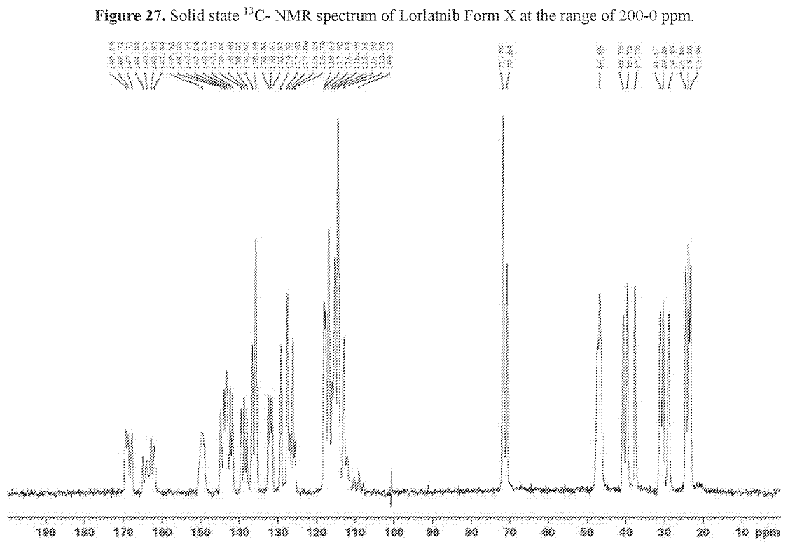

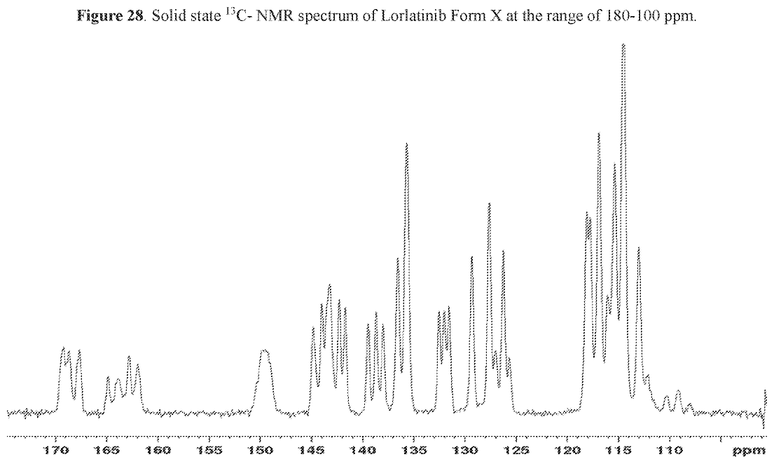

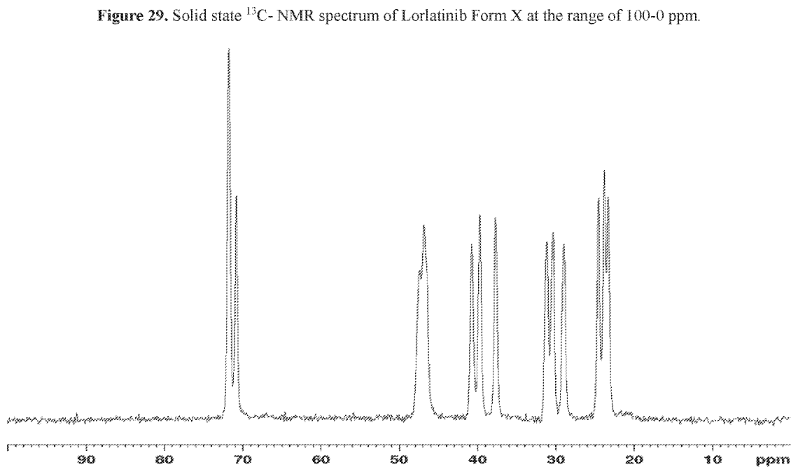

The present disclosure includes a crystalline form of Lorlatinib designated as Form X. The crystalline Form X of Lorlatinib can be characterized by data selected from one or more of the following: an XRPD pattern having peaks at 8.8, 11.0, 13.1, 17.6 and 22.4 degrees two theta±0.2 degrees two-theta; an XRPD pattern substantially as depicted in FIG. 19; a solid-state ¹³C NMR spectrum with signals at 37.7, 40.8, 71.8, 116.9 and 135.7 ppm±0.2 ppm; a solid state ¹³C NMR spectrum having the following chemical shift absolute differences from a peak at 135.7 ppm±2 ppm of 98.0, 94.9, 63.9, 18.8 and 0 ppm±0.1 ppm; a solid-state ¹³C NMR spectrum as depicted in FIG. 27 or 28 or 29; or a combination of these data.

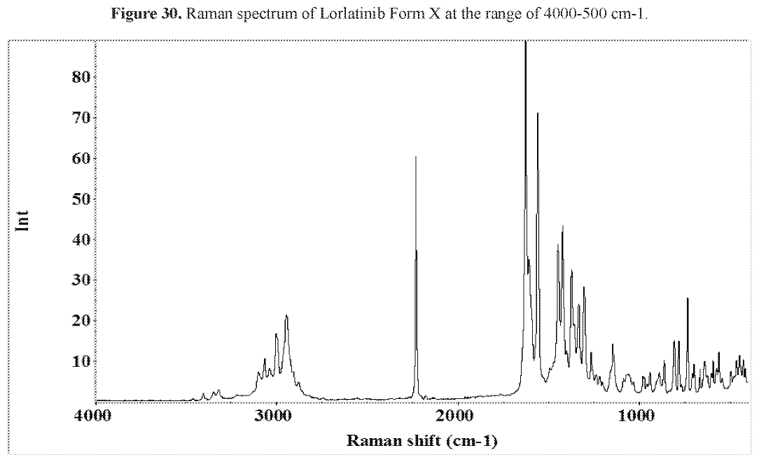

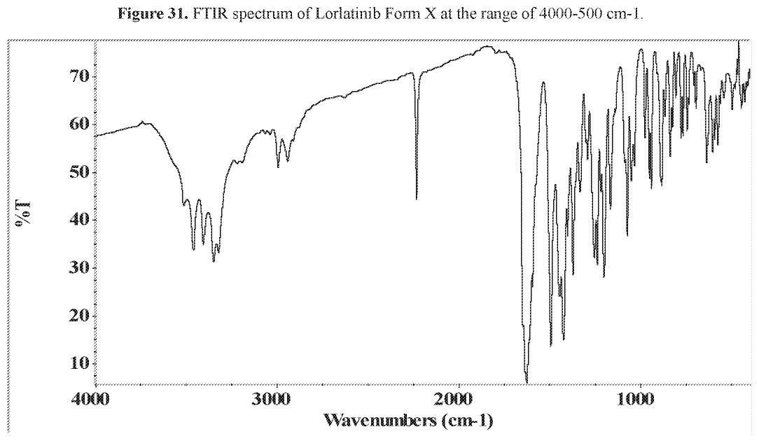

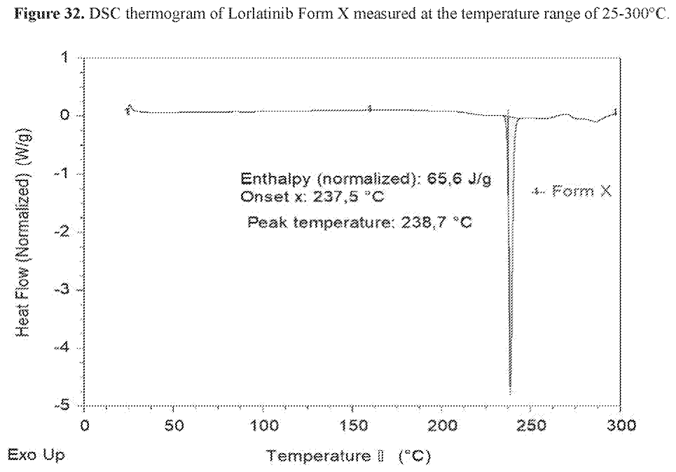

Crystalline Lorlatinib Form X may be further characterized by data selected from one or more of the following: an XRPD pattern having peaks at 8.8, 11.0, 13.1, 17.6 and 22.4 degrees two theta±0.2 degrees two-theta; and also having one, two or three additional peaks selected from 9.7, 12.3 and 17.0 degrees two theta±0.2 degrees two theta; FTIR spectrum having maxima of the characteristic bands at about 761.5, 821.4, 832.8, 2991.7, 3315.5, 3401.5 and 3454.1 cm⁻¹±2 cm⁻¹; FT Raman spectrum having maxima of the characteristic bands at about 1300.9, 1369.4, 1555.6, 1623.0 and 2999.4 cm⁻¹±2 cm⁻¹; DSC thermogram having onset peak temperature at about 237.5° C.±1° C.; an FTIR spectrum as depicted in FIG. 31; a Raman spectrum as depicted in FIG. 30; a DSC thermogram as depicted in FIG. 32; and combinations of these data.

In one embodiment Lorlatinib Form X can be an anhydrous form.

Crystalline Lorlatinib Form X may alternatively be characterized by an XRPD pattern having peaks at 8.8, 9.7, 11.0, 12.3, 13.1, 17.0, 17.6 and 22.4 degrees two theta±0.2 degrees two theta.

Crystalline Lorlatinib Form X may be characterized by each of the above characteristics alone/or by all possible combinations, e.g. XRPD pattern having peaks at 8.8, 11.0, 13.1, 17.6 and 22.4 degrees two theta±0.2 degrees two theta and an XRPD pattern as depicted in FIG. 19, and combinations thereof.

In any aspect or embodiment of the present disclosure, the crystalline Lorlatinib Form X described herein may be substantially free of any other solid state forms of Lorlatinib. In embodiments, crystalline Lorlatinib Form X according to any aspect or embodiment of the present invention contains about 20% (w/w) or less, about 10% (w/w) or less, about 5% (w/w) or less, about 2% (w/w) or less, about 1% (w/w) or less, or about 0% (w/w) of any other forms of Lorlatinib as measured, for example, by XRPD. Thus, according to any aspect or embodiment of the present disclosure, the crystalline form includes greater than about 80% (w/w), greater than about 90% (w/w), greater than about 95% (w/w), greater than about 98% (w/w), greater than about 99% (w/w), or about 100% of Form X of Lorlatinib. In embodiments, Lorlatinib Form X according to any aspect or embodiment of the present disclosure contains no detectable amounts of other solid state forms of Lorlatinib (for example as measured by XRPD).

In another embodiment of the present disclosure, crystalline Lorlatinib Form X is polymorphically pure.

In another embodiment of the present disclosure, crystalline Lorlatinib Form X is isolated.

As described above, depending on which other solid state it is compared with, Form X of Lorlatinib according to the present disclosure may have advantageous properties as described above. For example, Lorlatinib Form X is surprisingly thermodynamically stable.

In another embodiment Lorlatinib Form X is non-hygroscopic.

The above Lorlatinib Form X can be prepared by a process including crystallization of Lorlatinib in toluene. In embodiments the crystallization is carried out in substantially anhydrous conditions, for example the crystallization is carried out in the presence of: ≤1 wt %, ≤0.5 wt %, ≤0.25 wt %, ≤0.1 wt %.≤0.05 wt %, ≤0.02 wt %, ≤0.01 wt %, ≤0.005 wt %, ≤0.002 wt % water. Water may be removed or excluded by use of a Dean Stark trap.

In embodiments, the process includes using any one or a mixture of Lorlatinib solid state forms and/or any one or a mixture of Lorlatinib salts and their solid state forms thereof. If a Lorlatinib salt is used, the salt is converted to the free base form before crystallization. The free base form may be an anhydrous or a hydrate form, such as Form 2 or Form Epsilon; in embodiments the free base is a hydrate form.

In embodiments, 5-50 vol, in embodiments 10-30 vol, in embodiments 15-25 vol, and in other embodiments about 20 vol, of toluene is used.

In embodiments, Lorlatinib is dissolved in Toluene by heating to reflux.

In embodiments, the solution is kept under the above described conditions for about 1-5 hours, in embodiments for about 1-3 hours, and in other embodiments for about 2 hours, optionally with stirring.

In embodiments, prior to isolating the Lorlatinib Form X, the solution is cooled to a temperature of about 25 to about −5° C., in embodiments about 25 to about 0° C., in other embodiments to 20 to about 25° C., optionally with stirring.

In embodiments, isolation of Lorlatinib Form X may be done, for example, by filtering the resulting suspension and optionally drying. Drying is done by nitrogen or air or under vacuum. In embodiments, drying is performed at a temperature of about 20-80° C., in embodiments about 40-70° C., in embodiments about 50-70° C., in other embodiments about 60° C., when the drying is under vacuum (in embodiments at a reduced pressure of: 1-200 mbar, in embodiments 1-100 mbar, in embodiments 1-50 mbar, in other embodiments 1-20 mbar or about 10 mbar).

Alternatively, the present disclosure further includes a process for the preparation of Lorlatinib Form X including a solvent/antisolvent crystallization. The process includes:

a) providing a solution of Lorlatinib in polar aprotic solvent, in embodiments under heating;
b) crystallization with addition of n-Heptane, optionally with seeding
d) cooling, and optionally filtering and drying the powder.

Optionally, Lorlatinib used in stage a) can be prepared by conversion of Lorlatinib salt with a suitable base to obtain Lorlatinib. The Lorlatinib salt is basified with an aqueous inorganic base in the presence of the suitable organic solvent which is not miscible with water. Lorlatinib salt, such as Lorlatinib hydrochloride, is converted to Lorlatinib with suitable base and in polar aprotic solvent. The polar aprotic solvent is selected from ethyl acetate, DCM, Toluene, 2-methyl THF, n-butyl acetate and isobutyl acetate. In embodiments ethyl acetate is used. The suitable base can be selected from organic or inorganic bases, such as sodium hydroxide, potassium hydroxide, sodium carbonate, sodium bicarbonate, potassium carbonate and potassium bicarbonate, in embodiments, sodium hydroxide.

In embodiments, Lorlatinib produced in previous stage is isolated, for example, by extraction of the Lorlatinib free base into the organic solvent, followed by solvent evaporation. Lorlatinib may be extracted with ethyl acetate, in embodiments with addition of water. Optionally, ethyl acetate solution is partially evaporated by vacuum distillation to be used according to stage b) or optionally, full evaporation to obtain solid Lorlatinib to be used according to stage a).

Optionally, Lorlatinib used in stage a) can be also prepared by:
(i) deprotection of 10R)-(7-di-tert-butyloxycarbonylamino)-12-fluoro-2,10,16-trimethyl-15-oxo-10,15,16,17-tetrahydro-2H-8,4-(metheno)pyrazolo[4,3-h]-[2,5,11]benzoxadiazacyclotetradecine-3-carbonitrile in an organic solvent, such as a water-immiscible organic solvent (for example ethyl acetate), with an aqueous acid, for example hydrochloric acid;
(ii) collecting the organic phase;
(iii) basifying the organic phase, in embodiments an aqueous solution of a base, such as an aqueous solution of an inorganic base, for example an alkali metal hydroxide, such as sodium hydroxide;
(iv) collecting the organic phase; and
(v) optionally removing, or partially removing, the solvent from the organic phase to provide a solution of Lorlatinib free base, or Lorlatinib free base as a solid.

The polar aprotic solvent in stage a) can be selected from ethyl acetate, n-butyl-acetate, DCM, Toluene and 2-methyl THF. In embodiments, ethyl acetate is used.

In embodiments, the solution of Lorlatinib free base or Lorlatinib free base solid is used to prepare a crystallization solution comprising Lorlatinib and ethyl acetate, wherein the ethyl acetate is present at an amount of about 1-5 vol, in embodiments about 2-4 vol, in embodiments about 2.5-3.5 vol, and in yet other embodiments about 3 to about 3.5 vol of ethyl acetate, by heating to a temperature of about 40-80° C., in embodiments about 50-70° C., and in other embodiments to about 60° C., optionally with stirring.

In embodiments, crystallization is obtained by adding n-heptane according to stage b). In embodiments, 0.5 to 2 vol, in embodiments 1 to 1.5 vol, in yet other embodiments 1.2-1.5 vol of n-heptane is added dropwise, during 5-30 minutes, in embodiments 5-20 minutes, in yet other embodiments within 10 minutes, optionally with seeding. Optionally, about 0.1-10% by weight seeding with Form X is added. In embodiments 0.5-2.0% by weight, in yet other embodiments about 1% by weight seeding with Form X is added, optionally with stirring.

In embodiments, the suspension obtained in stage b) is further kept for 10-60 minutes, in embodiments for 10-30 minutes, in yet other embodiments for 20 minutes, optionally under stirring at a temperature of 40-80° C., in embodiments 50-70° C. and in yet other embodiments about 60° C. Optionally, another portion of n-heptane is added, in embodiments 1 vol to 10 vol, in embodiments 4 vol to 7 vol, in yet other embodiments 5-5.5 vol (referring to mass of starting material) was added drop wise. Time for addition may be during 10-120 minutes, in embodiments 10-60 minutes, in yet other embodiments during 30 minutes.

Optionally, the resulting suspension was kept at a temperature of 40-80° C., in embodiments 50-70° C. and in yet other embodiments at about 60° C. for a further 10-120 minutes, in embodiments about 30-80 minutes, in embodiments for about 60 minutes, optionally with stirring.

Prior to isolating Lorlatinib Form X, the suspension according to stage c) may be cooled to 10-30° C., in embodiments 20-30° C., in yet other embodiments about 20-25° C. Optionally the mixture may be further stirred for an additional 30-120 minutes, in embodiments for about 60 minutes.

Isolation of Lorlatinib Form X may be done for example by filtering the suspension, optionally with washing and optionally drying. Drying may be done by nitrogen or air or under vacuum. Drying may be performed at a temperature of about 40-60° C., in embodiments about 40-50° C., and in yet other embodiments about 45° C., when the drying is under vacuum (in embodiments at a reduced pressure of: 1-200 mbar, in embodiments 1-100 mbar, in embodiments 1-50 mbar, and in other embodiments 5-40 mbar or about 20 mbar).

The process may further include combining the crystalline Lorlatinib Form X with at least one pharmaceutically acceptable excipient to provide a pharmaceutical composition or formulation.

The process disclosure also relates to a crystalline form of Lorlatinib Form X, or a pharmaceutical composition or formulation thereof, which is obtainable by any embodiment of the process as described above.

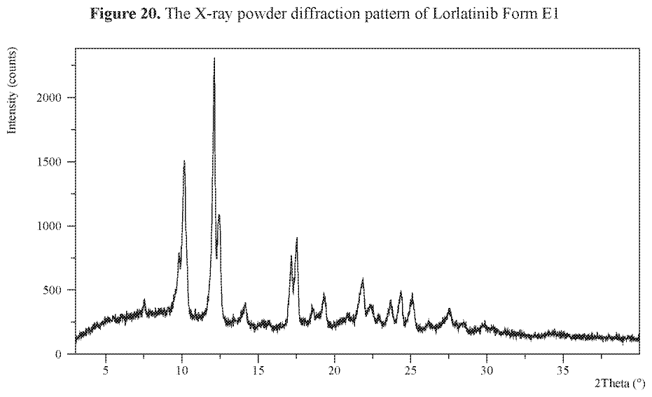

The present disclosure includes a crystalline form of Lorlatinib designated as Form E1. The crystalline Form E1 of Lorlatinib can be characterized by data selected from or more of the following: an XRPD pattern having peaks at 10.2, 12.1, 14.2, 17.2 and 21.9 degrees two theta±0.2 degrees two-theta; an XRPD pattern substantially as depicted in FIG. 20; or combinations of these data.

Crystalline Lorlatinib Form E1 may be further characterized by data selected from one or more of the following: an XRPD pattern having peaks at 10.2, 12.1, 14.2, 17.2 and 21.9 degrees two theta±0.2 degrees two-theta; and also having one, two, three or four additional peaks selected from 18.6, 23.7, 25.1 and 27.5 degrees two theta±0.2 degrees two theta; or combinations of these data. In one embodiment Lorlatinib Form E1 contains about 2-3% of water, in embodiments about 2.5%. Lorlatinib Form E1 can be characterized as hydrate form, in embodiments as a hemihydrate.

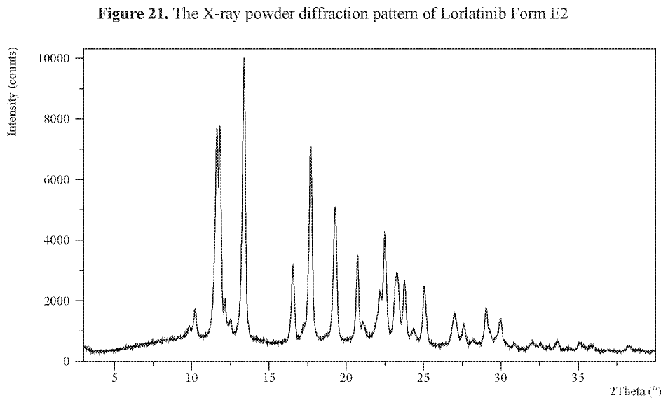

The present disclosure includes a crystalline form of Lorlatinib designated as Form E2. The crystalline Form E2 of Lorlatinib can be characterized by data selected from or more of the following: an XRPD pattern having peaks at 11.6, 13.4, 16.6, 20.7 and 22.5 degrees two theta±0.2 degrees two-theta; an XRPD pattern substantially as depicted in FIG. 21; or combinations of these data.

Crystalline Lorlatinib Form E2 may be further characterized by data selected from one or more of the following: an XRPD pattern having peaks at 11.6, 13.4, 16.6, 20.7 and 22.5 degrees two theta±0.2 degrees two-theta; and also having one, two, three or four additional peaks selected from 9.8, 17.7, 19.3 and 23.4 degrees two theta±0.2 degrees two theta; or combinations of these data. In one embodiment Lorlatinib Form E2 can be characterized as hydrate form.

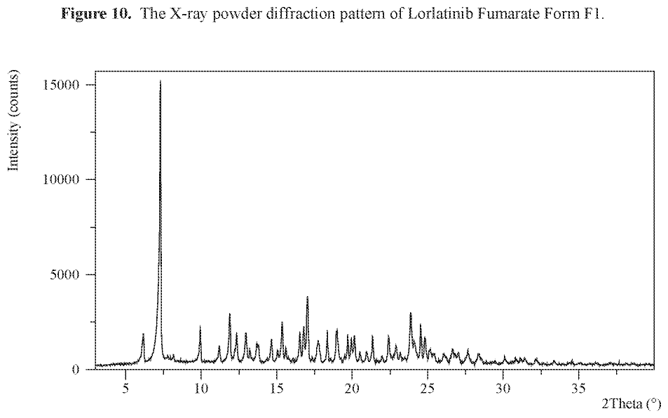

The present disclosure includes a crystalline form of Lorlatinib Fumarate designated as Form F1. The crystalline Form F1 of Lorlatinib Fumarate can be characterized by data selected from or more of the following: an XRPD pattern having peaks at 7.3, 11.9, 15.3, 17.0 and 23.9 degrees two theta±0.2 degrees two-theta; an XRPD pattern substantially as depicted in FIG. 10; or combinations of these data.

Crystalline Lorlatinib Fumarate Form F1 may be further characterized by data selected from one or more of the following: an XRPD pattern having peaks at 7.3, 11.9, 15.3, 17.0 and 23.9 degrees two theta±0.2 degrees two-theta; and also having one, two, three, four or five additional peaks selected from 10.0, 16.5, 16.8, 18.3 and 24.5 degrees two theta±0.2 degrees two theta; or combinations of these data.

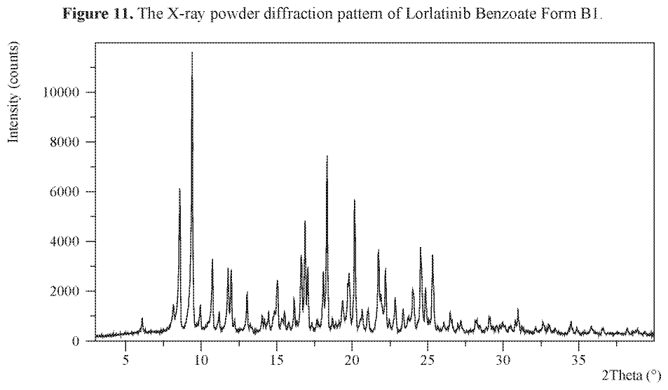

The present disclosure includes a crystalline form of Lorlatinib Benzoate designated as Form B1. The crystalline Form B1 of Lorlatinib Benzoate can be characterized by data selected from or more of the following: an XRPD pattern having peaks at 8.6, 9.4, 16.9, 18.3 and 20.2 degrees two theta±0.2 degrees two-theta; an XRPD pattern substantially as depicted in FIG. 11; or combinations of these data.

Crystalline Lorlatinib Benzoate Form B1 may be further characterized by data selected from one or more of the following: an XRPD pattern having peaks at 8.6, 9.4, 16.9, 18.3 and 20.2 degrees two theta±0.2 degrees two-theta; and also having one, two, three, four or five additional peaks selected from 10.8, 16.6, 21.7, 24.5 and 25.3 degrees two theta±0.2 degrees two theta; or combinations of these data.

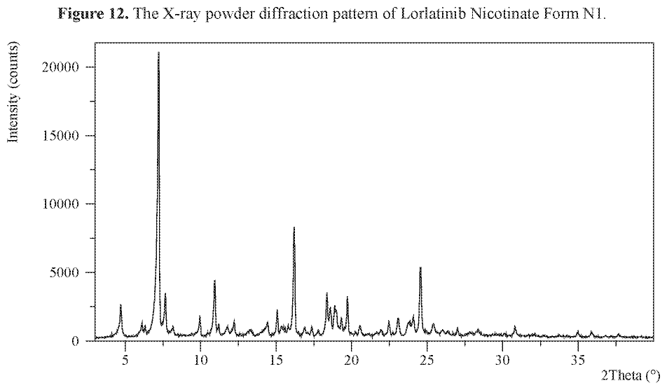

The present disclosure includes a crystalline form of Lorlatinib Nicotinate designated as Form N1. The crystalline Form N1 of Lorlatinib Nicotinate can be characterized by data selected from or more of the following: an XRPD pattern having peaks at 7.2, 10.9, 16.2, 18.4 and 24.6 degrees two theta±0.2 degrees two-theta; an XRPD pattern substantially as depicted in FIG. 12; or combinations of these data.

Crystalline Lorlatinib Nicotinate Form N1 may be further characterized by data selected from one or more of the following: an XRPD pattern having peaks at 7.2, 10.9, 16.2, 18.4 and 24.6 degrees two theta±0.2 degrees two-theta; and also having one, two, three, four or five additional peaks selected from 4.7, 7.7, 15.1, 18.9 and 19.7 degrees two theta±0.2 degrees two theta; or combinations of these data.

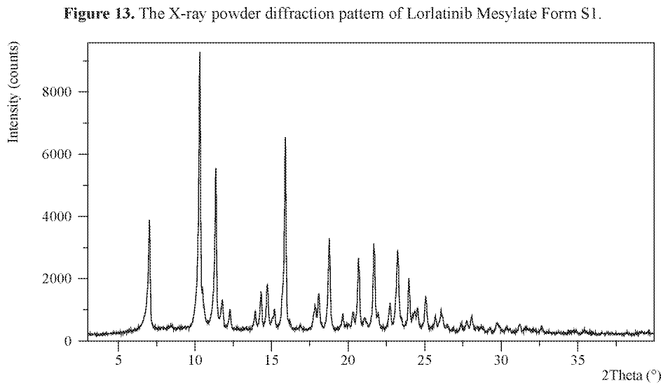

The present disclosure includes a crystalline form of Lorlatinib Mesylate designated as Form S1. The crystalline Form S1 of Lorlatinib Mesylate can be characterized by data selected from or more of the following: an XRPD pattern having peaks at 7.0, 10.3, 11.4, 15.9 and 18.8 degrees two theta±0.2 degrees two-theta; an XRPD pattern substantially as depicted in FIG. 13; or combinations of these data.

Crystalline Lorlatinib Mesylate Form S1 may be further characterized by data selected from one or more of the following: an XRPD pattern having peaks at 7.0, 10.3, 11.4, 15.9 and 18.8 degrees two theta±0.2 degrees two-theta; and also having one, two, three, four or five additional peaks selected from 14.7, 20.7, 21.7, 23.2 and 24.0 degrees two theta±0.2 degrees two theta; or combinations of these data.

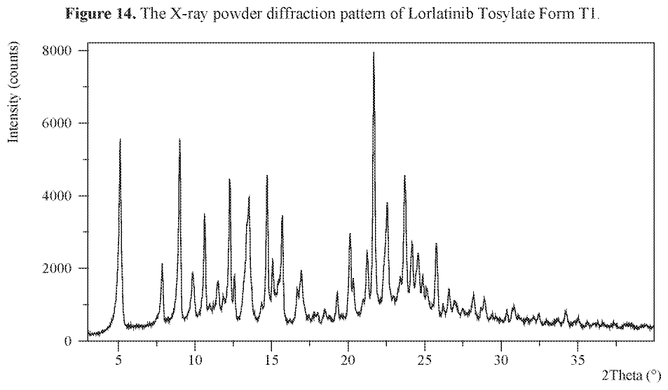

The present disclosure includes a crystalline form of Lorlatinib Tosylate designated as Form T1. The crystalline Form T1 of Lorlatinib Tosylate can be characterized by data selected from or more of the following: an XRPD pattern having peaks at 5.1, 9.0, 14.7, 21.7 and 23.7 degrees two theta±0.2 degrees two-theta; an XRPD pattern substantially as depicted in FIG. 14; or combinations of these data.

Crystalline Lorlatinib Tosylate Form T1 may be further characterized by data selected from one or more of the following: an XRPD pattern having peaks at 5.1, 9.0, 14.7, 21.7 and 23.7 degrees two theta±0.2 degrees two-theta; and also having one, two, three, four or five additional peaks selected from 10.6, 12.3, 13.5, 15.7 and 22.6 degrees two theta±0.2 degrees two theta; or combinations of these data.

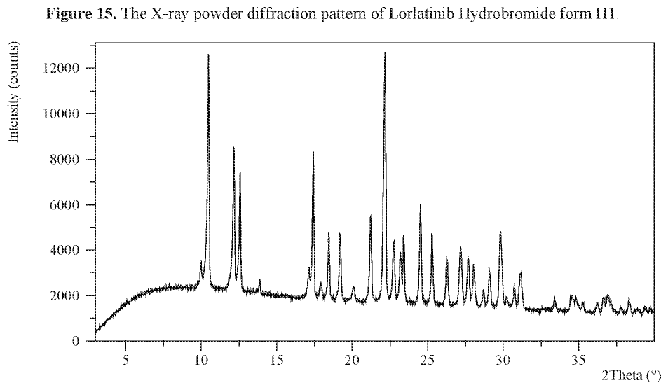

The present disclosure includes a crystalline form of Lorlatinib Hydrobromide designated as Form H1. The crystalline Form H1 of Lorlatinib Hydrobromide can be characterized by data selected from or more of the following: an XRPD pattern having peaks at 10.5, 12.2, 12.6, 17.4 and 22.2 degrees two theta±0.2 degrees two-theta; an XRPD pattern substantially as depicted in FIG. 15; or combinations of these data.

Crystalline Lorlatinib Hydrobromide Form H1 may be further characterized by data selected from one or more of the following: an XRPD pattern having peaks at 10.5, 12.2, 12.6, 17.4 and 22.2 degrees two theta±0.2 degrees two-theta; and also having one, two, three, four or five additional peaks selected from 21.2, 23.4, 24.5, 25.3 and 29.8 degrees two theta±0.2 degrees two theta; or combinations of these data.

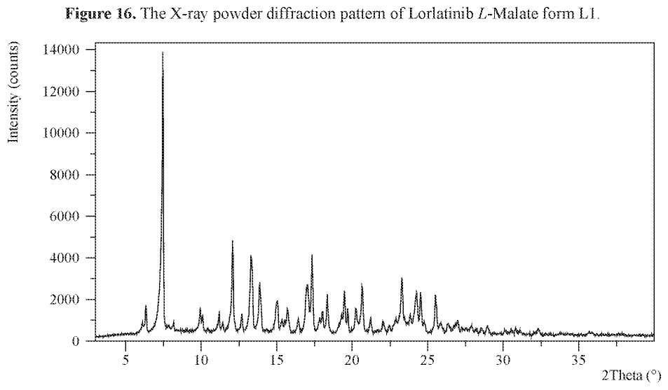

The present disclosure includes a crystalline form of Lorlatinib L-Malate designated as Form L1. The crystalline Form L1 of Lorlatinib L-Malate can be characterized by data selected from or more of the following: an XRPD pattern having peaks at 7.5, 12.1, 13.3, 17.3 and 23.3 degrees two theta±0.2 degrees two-theta; an XRPD pattern substantially as depicted in FIG. 16; or combinations of these data.

Crystalline Lorlatinib L-Maiate Form L1 may be further characterized by data selected from one or more of the following: an XRPD pattern having peaks at 7.5, 12.1, 13.3, 17.3 and 23.3 degrees two theta±0.2 degrees two-theta; and also having one, two, three, four or five additional peaks selected from 13.9, 17.1, 19.5, 20.7 and 24.3 degrees two theta±0.2 degrees two theta; or combinations of these data.

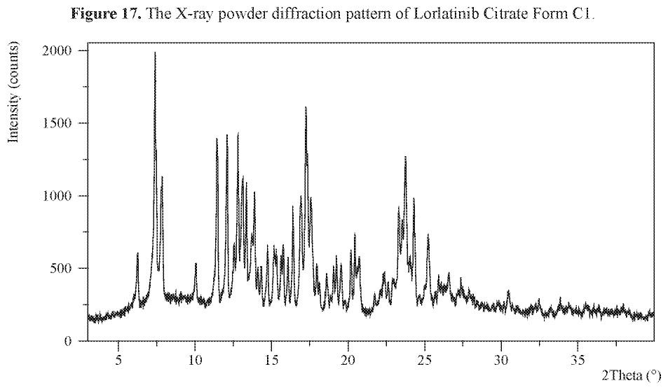

The present disclosure includes a crystalline form of Lorlatinib Citrate designated as Form C1. The crystalline Form C1 of Lorlatinib Citrate can be characterized by data selected from or more of the following: an XRPD pattern having peaks at 7.4, 11.4, 12.1, 12.8 and 17.2 degrees two theta±0.2 degrees two-theta; an XRPD pattern substantially as depicted in FIG. 17; or combinations of these data.

Crystalline Lorlatinib Citrate Form C1 may be further characterized by data selected from one or more of the following: an XRPD pattern having peaks at 7.4, 11.4, 12.1, 12.8 and 17.2 degrees two theta±0.2 degrees two-theta; and also having one, two, three, four or five additional peaks selected from 7.9, 13.1, 13.9, 23.8 and 24.3 degrees two theta±0.2 degrees two theta; or combinations of these data.

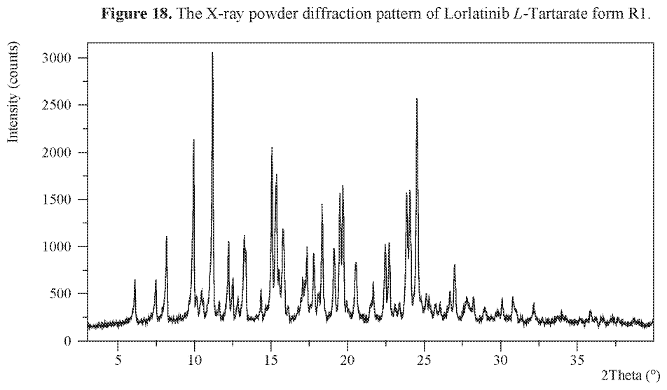

The present disclosure includes a crystalline form of Lorlatinib L-Tartarate designated as Form R1. The crystalline Form R1 of Lorlatinib L-Tartarate can be characterized by data selected from or more of the following: an XRPD pattern having peaks at 9.9, 11.2, 15.1, 15.4 and 24.5 degrees two theta±0.2 degrees two-theta; an XRPD pattern substantially as depicted in FIG. 18; or combinations of these data.

Crystalline Lorlatinib L-Tartarate Form R1 may be further characterized by data selected from one or more of the following: an XRPD pattern having peaks at 9.9, 11.2, 15.1, 15.4 and 24.5 degrees two theta±0.2 degrees two-theta; and also having one, two, three, four or five additional peaks selected from 18.3, 19.5, 19.7, 23.8 and 24.1 degrees two theta±0.2 degrees two theta; or combinations of these data.

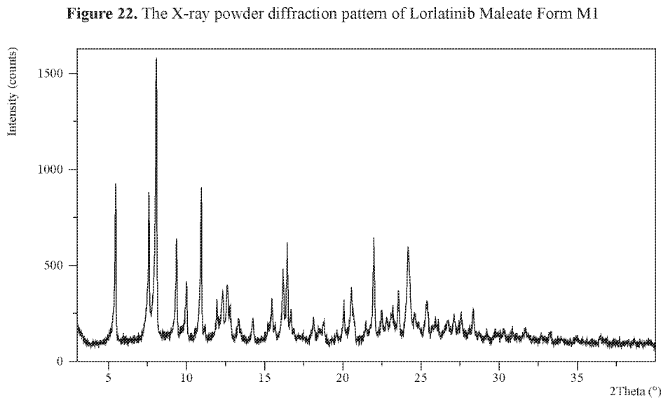

The present disclosure includes a crystalline form of Lorlatinib Maleate designated as Form M1. The crystalline Form M1 of Lorlatinib Maleate can be characterized by data selected from or more of the following: an XRPD pattern having peaks at 5.5, 8.1, 9.4, 10.9 and 14.3 degrees two theta±0.2 degrees two-theta; an XRPD pattern substantially as depicted in FIG. 22; or combinations of these data.

Crystalline Lorlatinib Maleate Form M1 may be further characterized by data selected from one or more of the following: an XRPD pattern having peaks at 5.5, 8.1, 9.4, 10.9 and 14.3 degrees two theta±0.2 degrees two-theta; and also having one, two, three, four or five additional peaks selected from 12.3, 12.6, 16.4, 22.0 and 24.2 degrees two theta±0.2 degrees two theta; or combinations of these data.

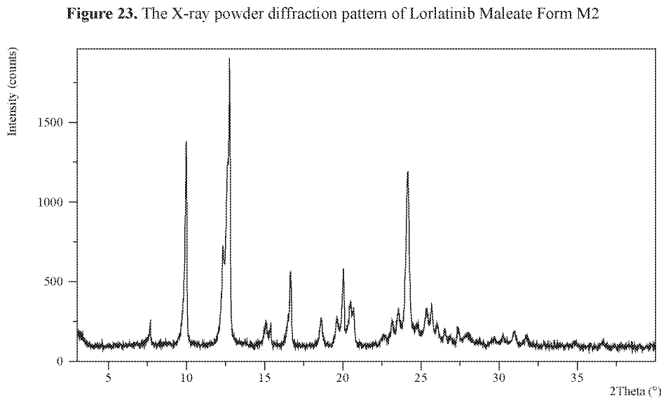

The present disclosure incudes a crystalline form of Lorlatinib Maleate designated as Form M2. The crystalline Form M2 of Lorlatinib Maleate can be characterized by data selected from or more of the following: an XRPD pattern having peaks at 7.7, 10.0, 12.8, 15.1 and 20.0 degrees two theta±0.2 degrees two-theta; an XRPD pattern substantially as depicted in FIG. 23; or combinations of these data.

Crystalline Lorlatinib Citrate Form M2 may be further characterized by data selected from one or more of the following: an XRPD pattern having peaks at 7.7, 10.0, 12.8, 15.1 and 20.0 degrees two theta±0.2 degrees two-theta; and also having one, two, three or four additional peaks selected from 7.7, 16.7, 18.6, 24.2 degrees two theta±0.2 degrees two theta; or combinations of these data. In another embodiment Lorlatinib Maleate Form M2 can be characterized as anhydrous form.

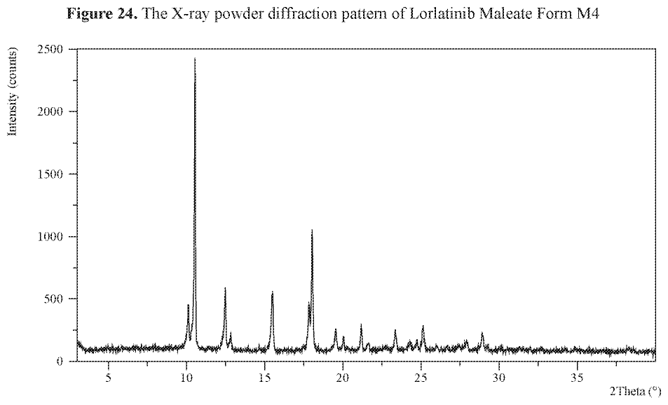

The present disclosure includes a crystalline form of Lorlatinib Maleate designated as Form M4. The crystalline Form M4 of Lorlatinib Maleate can be characterized by data selected from or more of the following: an XRPD pattern having peaks at 10.5, 12.5, 15.5, 18.0 and 19.5 degrees two theta±0.2 degrees two-theta; an XRPD pattern substantially as depicted in FIG. 24; or combinations of these data.

Crystalline Lorlatinib Maleate Form M4 may be further characterized by data selected from one or more of the following: an XRPD pattern having peaks at 10.5, 12.5, 15.5, 18.0 and 19.5 degrees two theta±0.2 degrees two-theta; and also having one, two, three or four additional peaks selected from 10.1, 21.2, 23.4, and 25.1 degrees two theta±0.2 degrees two theta; or combinations of these data.

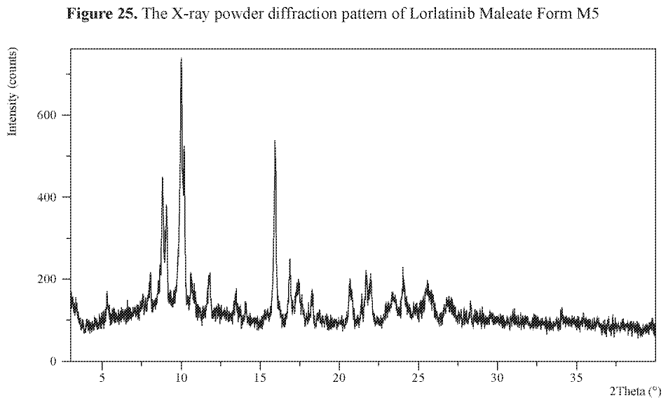

The present disclosure includes a crystalline form of Lorlatinib Maleate designated as Form M5. The crystalline Form M5 of Lorlatinib Maleate can be characterized by data selected from or more of the following: an XRPD pattern having peaks at 8.8, 11.8, 16.0, 16.9 and 21.7 degrees two theta±0.2 degrees two-theta; an XRPD pattern substantially as depicted in FIG. 25; or combinations of these data.

Crystalline Lorlatinib Maleate Form M5 may be further characterized by data selected from one or more of the following: an XRPD pattern having peaks at 8.8, 11.8, 16.0, 16.9 and 21.7 degrees two theta±0.2 degrees two-theta; and also having one, two, three or four additional peaks selected from 9.1, 10.0, 16.0 and 20.7 degrees two theta±0.2 degrees two theta; or combinations of these data.

The present disclosure also provides processes for the preparation of the solid state forms of Lorlatinib or Lorlatinib salts and their solid state forms thereof. The said process can include the processes set out in the examples herein below.

The present disclosure also provides uses of the solid state form of Lorlatinib, Lorlatinib salts and their solid state forms thereof of the present disclosure for preparing other solid state forms of Lorlatinib, other Lorlatinib salts and their solid state forms thereof.

The present disclosure also relates to the solid state forms of Lorlatinib, Lorlatinib salts, and their solid state forms thereof of the present disclosure for use in preparing other solid state forms of Lorlatinib and other salts of Lorlatinib and their solid state forms thereof.

The present disclosure further encompasses processes for preparing other solid state forms of Lorlatinib, other Lorlatinib salts and their solid state forms thereof. The said process includes preparing any one or a mixture of the Lorlatinib solid state forms, or Lorlatinib salts and their solid state forms thereof, according to the present disclosure, and converting it to Lorlatinib and solid state forms thereof or to Lorlatinib salts and solid state forms thereof. The conversion can be done, for example, by reacting the solid state form of Lorlatinib hydrochloride, described herein with a suitable base to obtain Lorlatinib, and optionally reacting it with an acid to obtain the corresponding acid-addition salt. Alternatively, the conversion to other Lorlatinib salts may be carried out by reacting the solid state of Lorlatinib with a suitable acid to obtain the desired Lorlatinib acid addition salt.

The present disclosure encompasses a process for preparing a pharmaceutically acceptable salt of Lorlatinib comprising reacting a solid state form of Lorlatinib according to the present disclosure with an acid. The process may further include combining the resulting pharmaceutically acceptable salt of Lorlatinib with at least one pharmaceutically acceptable excipient to produce a pharmaceutical formulation or dosage form.

In another aspect, the present disclosure encompasses the above described solid state forms of Lorlatinib, Lorlatinib salts and solid state forms thereof for use in the preparation of pharmaceutical compositions and/or formulations, in embodiments for use in medicine, such as for treatment of cancer.

In another aspect, the present disclosure encompasses the use of the above described solid state forms of Lorlatinib, Lorlatinib salts and solid state forms thereof for the preparation of pharmaceutical compositions and/or formulations, in embodiments for use in medicine, such as for treatment of cancer.

In yet another embodiment, the present disclosure encompasses pharmaceutical compositions including any one or a mixture of the solid state forms of Lorlatinib and Lorlatinib salts and solid state forms thereof.

In specific embodiment, the present disclosure encompasses pharmaceutical formulations including any one or a mixture of the solid state forms of Lorlatinib and Lorlatinib salts and solid state forms thereof and at least one pharmaceutically acceptable excipient.

The present disclosure further encompasses processes to prepare said pharmaceutical formulations of Lorlatinib and/or salts of Lorlatinib, by combining any one or a mixture of the above described salts and solid state forms of Lorlatinib, or pharmaceutical compositions including them, and at least one pharmaceutically acceptable excipient.

The present disclosure includes processes for preparing the above mentioned pharmaceutical compositions. The processes include combining any one or a mixture of the above crystalline polymorphs of Lorlatinib, Lorlatinib salts and/or combinations thereof of the present disclosure with at least one pharmaceutically acceptable excipient.

Pharmaceutical formulations of the present invention contain any one or a combination of the solid state forms of Lorlatinib and salt thereof of the present disclosure, particularly crystalline Lorlatinib forms Z, Gamma, U1, U2, Epsilon, X, E1 and E2. In embodiments, crystalline Lorlatinib Forms X and Epsilon are used. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye.

Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present invention include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present disclosure, active ingredient and any other solid excipients are dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present disclosure can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the administration route is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, in embodiments a powdered or granulated solid composition of the disclosure, within either a hard or soft shell. The shell can be made from gelatin and optionally contains a plasticizer such as glycerin and sorbitol, and an opacifying agent or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, but they are not subjected to a final tableting step.

A pharmaceutical formulation of Lorlatinib can be administered. Lorlatinib may be formulated for administration to a mammal, such as a human, by injection. Lorlatinib can be formulated, for example, as a viscous liquid solution or suspension, preferably a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The solid state forms of Lorlatinib, Lorlatinib salts and their solid state forms thereof as defined herein as well as the pharmaceutical compositions or formulations including them can be used as medicaments, in embodiments for treating non-small cell lung cancer, by administering a therapeutically effective amount of any of the solid state forms of the present disclosure, or at least one of the above pharmaceutical compositions or formulations, to a subject suffering from cancer or otherwise in need of the treatment.

The present disclosure also provides the uses of the solid state forms of Lorlatinib, Lorlatinib salts and their solid state forms thereof of the present disclosure, or at least one of the above pharmaceutical compositions or formulations, for the manufacture of medicaments for treating cancer, such as for treating non-small cell lung cancer.

Having described the disclosure with reference to certain preferred embodiments, other embodiments will become apparent to one skilled in the art from consideration of the specification. The disclosure is further illustrated by reference to the following examples describing in detail the preparation of the composition and methods of use of the disclosure. It will be apparent to those skilled in the art that many modifications, both to materials and methods, may be practiced without departing from the scope of the disclosure.

### Analytical Methods

### XRPD Method

X-ray Powder Diffraction was performed on an X-Ray powder diffractometer Philips X'Pert PRO; CuKα radiation (λ=1.54184 Å); X'Celerator detector with active length 2.122 degrees 2-theta; using the following scanning parameters: angle range: 3-40 deg., step size 0.0167, time per step 37 s, continuous scan.

### FTIR Method

FTIR measurement was recorded on Nicolet 6700 FT-IR spectrometer operating in the range 4000-400 cm⁻¹, equipped with KBr beamsplitter and DTGS detector. 16 scans were recorded at resolution of 4.0 cm⁻¹. Spectrum was recorded on Smart Orbit horizontal ATR accessory with diamond prism. Empty prism was used for background spectrum acquisition.

### FT Raman Method

Raman spectrum was recorded according to the following procedure: Powder sample was filled into 5 mm NMR tube and Raman spectrum was recorded on Nicolet 6700 FT-IR spectrometer with NXR FT-Raman module, equipped with 1064 nm Nd:YV04 excitation laser, CaF2 beam splitter and Ge detector. ¹³C-SS NMR METHOD

¹³C solid state NMR spectra was recorded with variable amplitude cross polarization, magic angle spinning and high power proton decoupling using a BRUKER Avance II+ spectrometer operating at 125.77 MHz and 303 K. A probe using 4 mm o.d. zirconia rotors was employed. The operation conditions were: contact time: 1 ms; recycle delay: 5 s; 2048 scans and spin rate of 18 kHz. Chemical shifts were referenced via a replacement sample of glycine (carboxyl carbon chemical shift assigned as 176.03 ppm relative to the signal of tetramethylsilane).

### DSC Method

DSC thermogram was measured using TA Instruments Discovery, DSC unit. About 1-3 mg of sample was weighted in pan, hermetically closed with the pin hole. Sample was purged with 50 ml/min of nitrogen flow and heated in the range of 25-300° C., using heating rate of 10° C./min.

### EXAMPLES

Lorlatinib can be obtained by any procedure described in the literature, for example using the syntheses procedure reported in U.S. Pat. Nos. 8,680,111 and 9,133,215 (Examples 2 and 132). Lorlatinib form 2 used in examples below can be prepared according to procedures described in U.S. Pat. No. 9,637,500. Amorphous Lorlatinib used in examples below can be prepared according U.S. Pat. No. 9,637,500 and according to Examples 5 and 6 described below. Lorlatinib Hydrochloride salt used as the starting material in Examples 1, 10 and 22 can be prepared according to Example 9 and according to procedure described in the J. Med. Chem. 2014, 57, 4720-4744. Any Lorlatinib polymorph, such as Form 2 or any of the forms described herein, can be used as the starting material to prepare clear solution in examples 11-21. The Lorlatinib free base used in Example 20 may be a hydrated form, such as Form 2 or Form Epsilon, and in some cases Form 2 can be used. Lorlatinib Maleate salt used as the starting material in examples 23-26 can be prepared according to example 27 and according to procedures described in International Publication No. WO 2017/175091 (example 1 for Lorlatinib Maleate Form 2 and example 2 for Lorlatinib Maleate Form 1).

### Example 1: Preparation of Lorlatinib Form Z and Form Gamma

1.9 grams of Lorlatinib hydrochloride salt (prepared according to Example 9) was dissolved in dichloromethane (20 mL), then water was added (20 mL) and pH was adjusted to 9-10 by addition of 10% solution of NaOH. The layers were separated and aqueous layer was extracted once again with dichloromethane (15 mL). Organic layers were combined, dried at anhydrous Na2SO4 and evaporated to dryness yielding 1.25 grams of residue of Lorlatinib. 1.25 grams of the residue was suspended in methanol (3.75 mL) and heated to 55° C. to dissolve. Then, water (2.5 mL) was added drop-wise into solution and crystallization was occurred. The obtained suspension was stirred at 55° C. for 30 minutes and then and left cool down slowly to room temperature for about 2-3 hours. At room temperature, water (1.25 mL) was added drop-wise and left stirring overnight. The resulting suspension was filtered off and wet product was divided into the following two drying procedures:

a. Drying at room temperature (open Petri dish at about 20-25° C., relative humidity about 30-40%) for 20 hours (yield: 423 mg, water content by KF 9.9%). The obtained product was analyzed by XRPD and the XRPD pattern is presented in FIG. 1.
b. Drying at 40° C. under vacuum (20 mbar) for 2 hours (yield: 480 mg, water content by KF 2.1%). The obtained product was analyzed by XRPD and the XRPD pattern is presented in FIG. 2.

### Example 2: Preparation of Lorlatinib Form Z

(10R)-(7-di-tert-butyloxy carbonyl amino)-12-fluoro-2,10,16-trimethyl-15-oxo-10,15,16,17-tetrahydro-2H-8,4-(metheno)pyrazolo[4,3-h]-[2,5,11]benzoxadiazacyclotetradecine-3-carbonitrile (3.68 grams, 6.07 mmol, prepared according to Example 7) was dissolved in ethyl acetate (18.4 mL) and 37% HCl was added dropwise while keeping the internal temperature below 28° C. The reaction mixture was stirred for 17 hours to complete Boc-deprotection. To the reaction mixture, water (40 ml) was added and layers were separated at pH<1. Then, to the aqueous layer, fresh ethyl acetate (40 mL) was added and pH was adjusted to pH 9.2 using saturated solution of Na₂CO₃. Layers were separated and organic layer was dried at anhydrous Na₂SO₄ and evaporated to dryness. The evaporated residue containing 0.8 grams of Lorlatinib was dissolved in methanol (1.6 mL), then water (1.6 mL) was added and reaction suspension was warmed to reflux temperature and stirred for 1 hour. The obtained solution was then cooled at room temperature for 2-3 hours and oiling out was occurred. The mixture was then warmed again to reflux temperature for about half an hour and left cool down slowly to room temperature overnight. Oiling out was occurred again and the mixture was stirred for one day at room temperature and crystallization was occurred. The resulting suspension was filtered off and dried at room conditions (open Petri dish at about 20-25° C. and 30-40% relative humidity) yielding 676 mg of crystalline Lorlatinib form Z (water content by KF 9.4%).

### Example 3: Preparation of Lorlatinib Form U1

50 mg of Lorlatinib (amorphous, prepared according to Example 5) was suspended in heptane (0.500 mL). The vial with magnet was tightly closed with a lid and left in sand bath at 40° C. with magnetic stirrer (700 rpm). After 24 hours the sample was filtered under vacuum and the obtained product was analyzed by XRPD and the XRPD pattern is presented in FIG. 3.

### Example 4: Preparation of Lorlatinib Form U2

50 mg of Lorlatinib (amorphous, prepared according to Example 5) was suspended in heptane (0.500 mL). The vial with magnet was tightly closed with a lid and left in cold laboratory (5° C.-10° C.) magnetic stirrer (700 rpm). After 24 hours sample was filtered under vacuum and the obtained product was analyzed by XRPD and the XRPD pattern is presented in FIG. 4.

### Example 5: Preparation of Amorphous Lorlatinib

5.06 grams of Lorlatinib (Form 2) was dissolved in dimethyl sulfoxide (30 mL) in round flask at room temperature. Solution was filtered and filtrate was transferred to petri dish. Solution was cooled in liquid nitrogen and the petri dish with frozen solution was put in freeze dryer at −40° C. for 4 hours and the frozen solution was transferred to the Lyophilizer.

The Lyophilisation program was set as following:
1. Freezing at −40° C. at atmospheric pressure for 4 hours
2. Main drying at −40° C. at 0.1 mbar for 24 hours
3. Final drying at 25° C. at 0.001 mbar for 24 hours
4. Final drying at 40° C. at 0.001 mbar for 24 hours
   The obtained pale yellow powder was analyzed by XRPD and the XRPD pattern is presented in FIG. 5.

### Example 6: Preparation of Amorphous Lorlatinib

Lorlatinib (5 grams, Form 2) was dissolved in acetone (150 mL) at room temperature. The prepared solution was spray dried using Büchi Mini Spray Dryer B-290, with following parameters: Inlet: 90° C., Outlet: 40-50° C., Aspirator: 100%, Pump: 25%. XRPD measurement for the obtained powder confirmed amorphous form content.

### Example 7: Preparation of (10R)-(7-di-tert-butyloxycarbonylamino)-12-fluoro-2,10,16-trimethyl-15-oxo-10,15,16,17-tetrahydro-2H-8,4-(metheno)pyrazolo[4,3-h]-[2,5,11]benzoxadiazacyclotetradecine-3-carbonitrile

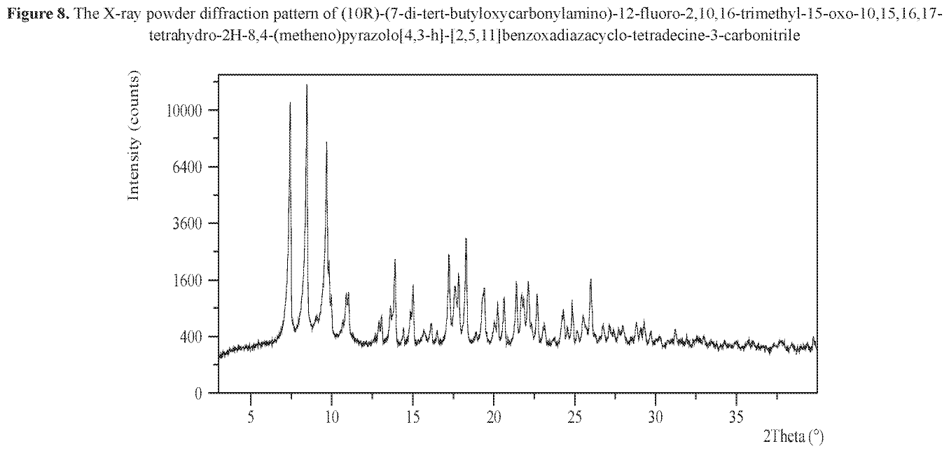

Potassium acetate (2.18 grams, 22.18 mmol) and Pd(OAc)₂ (131 mg, 0.58 mmol) were suspended in t-AmOH (125 mL) under argon atmosphere. Then water (0.131 mL, 7.27 mmol) and cataCXium A (Di(1-adamantyl)-n-butylphosphine, 417 mg, 1.16 mmol) were added and reaction mixture was stirred for 15 minutes at ambient temperature. Meanwhile, a solution of 2-[(1R)-1-{[5-bromo-2-(di-tert-butyloxycarbonylamino)pyridin-3-yl]oxy}-ethyl]-N-[(5-cyano-1-methyl-1H-pyrazol-3-yl)methyl]-4-fluoro-N-methylbenzamide (5 g, 7.27 mmol) in t-AmOH (25 mL) was prepared and an amount of 20% solution of 2-[(1R)-1-{[5-bromo-2-(di-tert-butyloxycarbonylamino)pyridin-3-yl]oxy}-ethyl]-N-[(5-cyano-1-methyl-1H-pyrazol-3-yl)methyl]-4-fluoro-N-methylbenzamide were added to mixture of base, catalyst and ligand. Resulting reaction mixture was heated to reflux for 0.5 hour and then remaining solution of 2-[(1R)-1-{[5-bromo-2-(tert-butyloxy-carbonylamino)pyridin-3-yl]oxy}-ethyl]-N-[(5-cyano-1-methyl-1H-pyrazol-3-yl)methyl]-4-fluoro-N-methylbenzamide was added dropwise over a period of 5 hours. The reaction mixture was heated for next 21 hours, then cooled and filtered through Celite to remove insoluble materials. The filtrate was evaporated to oil and then acetonitrile (25 mL) was added. Crystallization occurred and suspension was stirred overnight at ambient temperature and 1 hour on ice bath. Crystals were filtered off, washed with acetonitrile (10 mL) and dried in vacuum at 45° C. for 2 hours yielding 4.15 grams of (10R)-(7-di-tert-butyloxycarbonylamino)-12-fluoro-2,10,16-trimethyl-15-oxo-10,15,16,17-tetrahydro-2H-8,4-(metheno)pyrazolo[4,3-h]-[2,5,11]benzoxadiazacyclotetradecine-3-carbonitrile. The XRPD of the obtained product is presented in FIG. 8. This compound can be prepared also according to the procedure described in U.S. Pat. No. 9,637,500 (Example 4, step 2).

### Example 8. Preparation of Lorlatinib Mixture of Forms U1 and U2

100 mg of Lorlatinib (amorphous, prepared from lyophilisation) was suspended in mixture of heptane (1 mL) and methyl ethyl ketone (50 μL). The vial with magnet was tightly closed with a lid and left at room temperature on magnetic stirrer (700 rpm). After 24 hours sample was filtered under vacuum and XRPD measurement confirmed mixture of form U1 and form U2 was obtained.

### Example 9. Preparation of Lorlatinib Hydrochloride Salt

(10R)-(7-di-tert-butyloxycarbonylamino)-12-fluoro-2,10,16-trimethyl-15-oxo-10,15,16,17-tetrahydro-2H-8,4-(metheno)pyrazolo[4,3-h]-[2,5,11]benzoxadiazacyclotetradecine-3-carbonitrile (3 g, 4.95 mmol, prepared according to Example 7) was suspended in ethyl acetate (15 mL) and 37% HCl (3.25 mL) was added dropwise over 10 minutes. The reaction mixture was stirred for 18 hours at room temperature and product was crystalized. The obtained suspension was then cooled and stirred at 0-5° C. for 1 hour, filtrated and wet product was washed with ethyl acetate (6 mL) and dried under vacuum at 45° C. for 2 hours yielding 1.55 grams of Lorlatinib hydrochloride salt. Lorlatinib hydrochloride salt can also be prepared according to the procedure described in U.S. Pat. No. 9,637,500 (Example 4, step 3) and according to the procedure described in J. Med. Chem. 2014, 57, 4720-4744.

### Example 10. Preparation of Lorlatinib Form Epsilon

1.3 grams of Lorlatinib HCl salt (prepared according to Example 9) was dissolved in dichloromethane (15 mL), water was added (15 mL) and pH was adjusted to 9-10 by addition of 10% solution of NaOH. The layers were separated and aqueous layer was extracted once again with dichloromethane (15 mL). Organic layers were combined and evaporated to dryness yielding 1.16 grams of residue of Lorlatinib free base. The residue was dissolved in methanol (3.5 mL) and stirred at room temperature and after several minutes crystallization was observed. The obtained suspension was stirred at room temperature for additional 2 hours, then cooled at 0-5° C. and stirred for next 2 hours. The resulting suspension was filtered off and dried at 45° C., vacuum (10 mbar) during 3 hours, yielding 511 mg of crystalline Lorlatinib Form Epsilon, as confirmed by XRPD shown in FIG. 9 (water content by KF 3.36%).

### Example 11. Preparation of Lorlatinib Fumarate Form F1

Lorlatinib (200 mg) was suspended in 2 mL of 1-butanol. Suspension was heated to 90° C. and stirred until full dissolution was observed. Solution containing 62 mg of fumaric acid and 100 μL of water in 2 mL of 1-butanol was added dropwise to the hot solution of Lorlatinib. Solution was cooled to RT and precipitation occurred. Suspension was stirred at RT for additional 30 minutes. The obtained solid was filtered, washed three times with 2 mL of n-heptane and dried on air. The obtained solid corresponds to Lorlatinib fumarate form F1 as analyzed by XRPD and the XRPD pattern is shown in FIG. 10.

### Example 12. Preparation of Lorlatinib Benzoate Form B1

Lorlatinib (200 mg) was suspended in 2 mL of 1-butanol. Suspension was heated to 90° C. and stirred until full dissolution was observed. Solution containing 64 mg of benzoic acid in 2 mL of 1-butanol was added dropwise to the hot solution of Lorlatinib. Solution was cooled to RT and precipitation occurred. Suspension was stirred at RT for additional 30 minutes. Solids were filtered, washed three times with 2 mL of n-heptane and dried on air. The obtained solid corresponds to Lorlatinib benzoate form B1 as analyzed by XRPD and the XRPD pattern is shown in FIG. 11.

### Example 13. Preparation of Lorlatinib Nicotinate Form N1

Lorlatinib (200 mg) was suspended in 2 mL of 1-butanol. Suspension was heated to 90° C. and stirred until full dissolution was observed. Solution containing 64 mg of nicotinic acid and 200 μL of water in 2 mL of 1-butanol was added dropwise to the hot solution of Lorlatinib. Solution was cooled to RT and precipitation occurred. Suspension was stirred at RT for additional 30 minutes. Solids were filtered, washed three times with 2 mL of n-heptane and dried on air. The obtained solid corresponds to Lorlatinib nicotinate form N1 as analyzed by XRPD and the XRPD pattern is shown in FIG. 12.

### Example 14. Preparation of Lorlatinib Mesylate Form S1

Lorlatinib (200 mg) was suspended in 2 mL of 1-butanol. Suspension was heated to 90° C. and stirred until full dissolution was observed. Solution containing 73 mg of 70% w/w water solution of methanesulfonic acid in 2 mL of 1-butanol was added dropwise to the hot solution of Lorlatinib. Solution was cooled to RT, 5 mL of heptane was added and precipitation occurred. Suspension was stirred at RT for additional 30 minutes. Solids were filtered, washed three times with 2 mL of n-heptane and dried on air. The obtained solid corresponds to Lorlatinib mesylate form S1 as analyzed by XRPD and the XRPD pattern is shown in FIG. 13.

### Example 15. Preparation of Lorlatinib Tosylate Form T1

Lorlatinib (200 mg) was suspended in 2 mL of 1-butanol. Suspension was heated to 90° C. and stirred until full dissolution was observed. Solution containing 92 mg of p-toluenesulfonic acid in 2 mL of 1-butanol was added dropwise to the hot solution of Lorlatinib. Solution was cooled to RT and precipitation occurred. Suspension was stirred at RT for additional 30 minutes. Solids were filtered, washed three times with 2 mL of n-heptane and dried on air. The obtained solid corresponds to Lorlatinib tosylate form T1 as analyzed by XRPD and the XRPD pattern is shown in FIG. 14.

### Example 16. Preparation of Lorlatinib Hydrobromide Form H1

Lorlatinib (200 mg) was suspended in 2 mL of 1-butanol. Suspension was heated to 90° C. and stirred until full dissolution was observed. Solution containing 90 mg of 48% w/w water solution of hydrobromic acid in 2 mL of 1-butanol was added dropwise to the hot solution of Lorlatinib. Solution was cooled to RT and precipitation occurred. Suspension was stirred at RT for additional 30 minutes. Solids were filtered, washed three times with 2 mL of n-heptane and dried on air. The obtained solid corresponds to Lorlatinib hydrobromide form H1 as analyzed by XRPD and the XRPD pattern is shown in FIG. 15.

### Example 17. Preparation of Lorlatinib L-Malate Form L1

Lorlatinib (200 mg) was suspended in 2 mL of 1-butanol. Suspension was heated to 90° C. and stirred until full dissolution was observed. Solution containing 72 mg of L-malic acid in 2 mL of 1-butanol was added dropwise to the hot solution of Lorlatinib. Solution was cooled to RT and precipitation occurred. Suspension was stirred at RT for additional 30 minutes. Solids were filtered, washed three times with 2 mL of n-heptane and dried on air. The obtained solid corresponds to Lorlatinib L-malate form L1 as analyzed by XRPD and the XRPD pattern is shown in FIG. 16.

### Example 18. Preparation of Lorlatinib Citrate Form C1

Lorlatinib (1000 mg) was suspended in 10 mL of 1-butanol. Suspension was heated to 90° C. and stirred until full dissolution was observed. Solution containing 515 mg of citric acid in 10 mL of 1-butanol was added dropwise to the hot solution of Lorlatinib. Solution was cooled to 0° C. and precipitation occurred. Suspension was stirred at 0° C. for additional 30 minutes. Solids were filtered, washed three times with 5 mL of n-heptane and dried on air. The obtained solid corresponds to Lorlatinib citrate form C1 as analyzed by XRPD and XRPD pattern is shown in FIG. 17.

### Example 19. Preparation of Lorlatinib L-Tartarate Form R1

Lorlatinib (200 mg) was suspended in 2 mL of 1-butanol. Suspension was heated to 90° C. and stirred until full dissolution was observed. Solution containing 80 mg of L-tartaric acid and 200 μL of water in 2 mL of 1-butanol was added dropwise to the hot solution of Lorlatinib. Solution was cooled to RT and precipitation occurred. Suspension was stirred at RT for additional 30 minutes. Solids were filtered, washed three times with 2 mL of n-heptane and dried on air. The obtained solid corresponds to Lorlatinib L-tartarate form R1 as analyzed by XRPD and XRPD pattern is shown in FIG. 18.

### Example 20. Preparation of Lorlatinib Form X

500 mg of Lorlatinib base was dissolved in 10 ml of toluene by heating to 110° C. in the round flask equipped with Dean Stark apparatus. (Dean Stark apparatus is a piece of laboratory glassware used to collect water). The solution was stirred for 2 hours at 110° C. Then, the solution was cooled to room temperature by stirring. The obtained suspension was filtrated and the isolated solid was dried at 60° C. under vacuum for 3 hours. The obtained solid corresponds to Lorlatinib form X as was analyzed by XRPD and XRPD pattern is shown in FIG. 19.

### Example 21. Preparation of Lorlatinib Form E1

Sample of Lorlatinib base Form 2 was exposed to methanol vapours for 5 days. XRPD analysis showed that it was Form E1 of Lorlatinib base. XRPD result is given in FIG. 20.

### Example 22. Preparation of Lorlatinib Form E2

0.7 grams of Lorlatinib HCl salt was dissolved in dichloromethane (8.6 mL), water was added (8.6 mL) and pH was adjusted to 9-10 by addition of 10% solution of NaOH. The layers were separated and aqueous layer was extracted once again with dichloromethane (5.7 mL). Organic layers were combined and evaporated at temp of about 40° C. to dryness yielding 0.69 grams of residue of Lorlatinib free base. The residue was dissolved in methanol (1.8 mL) and stirred at room temperature and after several minutes crystallization was observed. The obtained suspension was stirred at room temperature for additional 1.5 hours, then cooled at 0-5° C. and stirred for more 2 hours. The resulting suspension was filtered off and dried at 45° C. under vacuum (10 mbar) for 3 hours, yielding 457 mg of crystalline Lorlatinib base Form E2 as was analyzed by XRPD and XRPD pattern is shown in FIG. 21.

### Example 23. Preparation of Lorlatinib Maleate Form M1

50 mg of Lorlatinib maleate was dissolved in 2 mL of methanol by heating on a hot plate at reflux in vial (at about 65° C.). Vial was cooled and tightly closed with a lid and left at ambient conditions. After 2 days, the product was filtered under vacuum and the obtained solid corresponds to Lorlatinib Maleate Form M1 as was analyzed by XRPD and XRPD pattern is shown in FIG. 22.

### Example 24. Preparation of Lorlatinib Maleate Form M2

100 mg of Lorlatinib maleate Form 1 (obtained according to example 27) was suspended in 5 mL of methyl-iso-butyl ketone by heating on a hot plate at reflux in vial (about 115° C.). Suspension was cooled to room temperature and the suspension was filtered under vacuum. The obtained solid corresponds to Lorlatinib Maleate Form M2 as was analyzed by XRPD and XRPD pattern is shown in FIG. 23.

### Example 25. Preparation of Lorlatinib Maleate Form M4

50 mg of Lorlatinib maleate Form 1 (obtained according to Example 27) was suspended in 5 mL of water by heating on a hot plate at reflux in vial. Suspension was cooled to room temperature and suspension was filtered under vacuum. The obtained solid corresponds to Lorlatinib Maleate Form M4 as was analyzed by XRPD and XRPD pattern is shown in FIG. 24.

### Example 26. Preparation of Lorlatinib Maleate Form M5

100 mg of Lorlatinib maleate was dissolved in 1 mL of 2-propanol/water mixture (1/1, V/V) by heating on a hot plate at reflux in vial (at about 85° C.). Vial was cooled to room temperature and then tightly closed with a lid and left at ambient conditions. After 1 day precipitation was filtered under vacuum. The obtained solid corresponds to Lorlatinib Maleate Form M5 as was analyzed by XRPD and XRPD pattern is shown in FIG. 25.

### Example 27. Preparation of Lorlatinib Maleate Form 1 (Described in WO 2017/175091)

10 grams of Lorlatinib base Form 2 was dissolved in 1-butanol (100 ml) at 90° C. Into the hot solution 1.1 eq of maleic acid was added. The added maleic acid solution was obtained by dissolving maleic acid (3.1 grams) in 2 butanol in concentration of 100 g/1. The hot solution was cooled down to room temperature and crystallization occurred. The suspension was stirred for 1 hour and filtrated off. Obtained material was washed 3 times with 20 ml of n-heptane and dried at 40° C. under vacuum over the night. XRPD analysis showed it was Form 1 of Lorlatinib Maleate. XRPD result is given in FIG. 26.

### Example 28. Preparation of Lorlatinib Form X

1.32 grams of (10R)-(7-di-tert-butyloxycarbonylamino)-12-fluoro-2,10,16-trimethyl-15-oxo-10,15,16,17-tetrahydro-2H-8,4-(metheno)pyrazolo[4,3-h]-[2,5,11]benzoxadiazacyclotetradecine-3-carbonitrile was suspended in ethyl-acetate (10 ml) and hydrochloric acid, 36% (0.86 ml) was added. The obtained mixture was then heated at 50° C. and stirred for 2 hours (until completion of deprotection reaction). The obtained suspension was then cooled at room temperature, then water (40 ml) was added and stirred for 10 minutes. Layers were separated and aqueous layer additionally washed with ethyl-acetate (5.3 ml). Into the washed aqueous layer, fresh ethyl-acetate (13.2 ml) was added and pH value adjusted at 10.3 by addition of 10% NaOH solution. The layers were separated and aqueous layer additionally extracted with ethyl-acetate (13.2 ml). The organic layers were combined and washed with water (13.6 ml). The obtained ethyl-acetate solution was concentrated by vacuum distillation to volume about 3.6 ml and heated at 60° C. n-Heptane (1.7 ml) was the added dropwise during 10 minutes. Optionally, seeding of Lorlatinib form X (8 mg) can be added. The obtained suspension was stirred for 20 minutes at 60° C. and additional amount of n-heptane (6.4 ml) was added during 30 minutes. The suspension was additionally stirred at 60° C. for 1 hour, then cooled at 20-25° C. and stirred for 1 hour. The product was filtered off, washed with n-heptane (3.6 ml) and dried in vacuum dryer (20 mbar, 45° C.) for 3 hours to obtain Lorlatinib form X (0.7 grams).

1. A crystalline form of Lorlatinib designated as Form Gamma, characterized by data selected from one or more of the following:
a. an X-ray powder diffraction pattern having peaks at 6.7, 8.9, 10.4, 12.3 and 14.3 degrees two theta±0.2 degrees two-theta and absence of peaks at 7.1, 8.4 and 15.6 degrees two-theta±0.2 degrees 2-theta;
b. an XRPD pattern as depicted in FIG. 2;
c. a combination of the two of the above.

2. The crystalline Form Gamma of Lorlatinib according to claim 1, which is a hydrate.

3. A crystalline form of Lorlatinib designated as Form X, characterized by data selected from one or more of the following:
a. an X-ray powder diffraction pattern having peaks at 8.8, 11.0, 13.1, 17.6 and 22.4 degrees two theta±0.2 degrees two-theta;
b. an XRPD pattern as depicted in FIG. 19;
c. a solid-state ¹³C NMR spectrum with signals at 37.7, 40.8, 71.8, 116.9 and 135.7 ppm±0.2 ppm;
d. a solid state ¹³C NMR spectrum having the following chemical shift absolute differences from a peak at 135.7 ppm±2 ppm of 98.0, 94.9, 63.9, 18.8 and 0 ppm±0.1 ppm;
e. a solid state ¹³C-NMR spectrum as depicted in FIG. 27 or 28 or 29; and
f. a combination of any two or more of the above.

4. The crystalline Form X of Lorlatinib according to claim 3, characterized by an XRPD pattern having peaks at 8.8, 11.0, 13.1, 17.6 and 22.4 degrees two theta±0.2 degrees two-theta, and also further characterized by one or more of the following:
a. having one, two or three additional XRPD peaks selected from 9.7, 12.3 and 17.0 degrees two theta±0.2 degrees two theta;
b. FTIR spectrum having maxima of the characteristic bands at about 761.5, 821.4, 832.8, 2991.7, 3315.5, 3401.5 and 3454.1 cm⁻¹±2 cm⁻¹;
c. FT Raman spectrum having maxima of the characteristic bands at about 1300.9, 1369.4, 1555.6, 1623.0 and 2999.4 cm⁻¹±2 cm⁻¹;
d. DSC thermogram having onset peak temperature at about 237.5° C.±1° C.;
e. a FTIR spectrum as depicted in FIG. 31;
f. a Raman spectrum as depicted in FIG. 30;
g. a DSC thermogram as depicted in FIG. 32;
h. and combinations of these data.

5. A crystalline form of Lorlatinib designated as Form Epsilon, characterized by data selected from one or more of the following:
a. an X-ray powder diffraction pattern having peaks at 6.4, 10.7, 9.0, 13.1 and 13.6 degrees two theta±0.2 degrees two-theta;
b. an XRPD pattern as depicted in FIG. 9;
c. a solid state ¹³C-SS NMR spectrum having characteristic peaks at 21.1, 38.3, 41.2, 115.2 and 132.5 ppm±0.2 ppm;
d. a solid state ¹³C-SS NMR spectrum having the following chemical shift absolute differences from a reference peak at 21.1 ppm±2 ppm of 0, 17.2, 20.1, 94.1 and 111.4 ppm±0.1 ppm.
e. a solid state ¹³C-SS NMR spectrum as depicted in FIG. 27 or 28 or 29; and
f. a combination of any two or more of the above.

6. The crystalline Form Epsilon of Lorlatinib according to claim 5, characterized by an XRPD pattern having peaks at 6.4, 10.7, 9.0, 13.1 and 13.6 degrees two theta±0.2 degrees two-theta and also further characterized by one or more of the following:
a. having one, two, three, four or five additional XRPD peaks selected from 7.7, 8.2, 20.7, 21.7 and 23.7 degrees two theta±0.2 degrees two theta;
b. having an absence of at least one, and preferably both, of the peaks at 6.7 and 7.2±0.2 degrees two-theta.
c. and combinations of these data.

7. A pharmaceutical composition comprising a crystalline form according to claim 1.

8. Use of a crystalline form according to claim 1 in the manufacture of a pharmaceutical composition and/or formulation.

9. A pharmaceutical formulation comprising a crystalline form according to claim 1, and at least one pharmaceutically acceptable excipient.

10. A process for preparing a pharmaceutical formulation comprising combining a crystalline form according to claim 1, with at least one pharmaceutically acceptable excipient.

11. The crystalline form according to claim 1, for use as a medicament.

12. The crystalline form, according to claim 1, for use in the treatment of cancer.

13. A method of treating cancer, comprising administering a therapeutically effective amount of a crystalline form according to claim 1, to a subject suffering from cancer.

14. The crystalline form, according to claim 1, for use in the manufacture of medicament for treatment of lung cancer.

15. A process for preparing Lorlatinib Form X according to claim 3, comprising crystallization of Lorlatinib in toluene.

16. A process for preparing Lorlatinib Form X according to claim 3, comprising the steps of:
a) providing a solution of Lorlatinib in polar aprotic solvent, preferably under heating;
b) crystallizing with addition of n-heptane, optionally with seeding; and
c) cooling, and optionally filtering; and drying.

17. A process according to claim 16, wherein the polar aprotic solvent is selected from ethyl acetate, n-butyl-acetate, dichloromethane, toluene and 2-methyl tetrahydrofuran, preferably ethyl acetate, and preferably wherein the polar aprotic solvent is used in an amount of about 1-5 volumes.

18. A process according to claim 16, wherein the n-heptane is used in an amount of 0.5 to 2 vol of n-heptane.

19. A process for preparing Lorlatinib Form Epsilon according to claim 5, comprising crystallization of Lorlatinib in methanol.

20. A process according to claim 19, comprising crystallization of a solution of Lorlatinib in methanol at about 20° C. to about 25° C. and optionally cooling and isolating Lorlatinib Form Epsilon.
